SEQUENCE LISTING (4 sequences)

SEQ ID NO: 1 - protein, 123 aa, synthetic construct
KQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 2 - protein, 124 aa, synthetic construct
MKQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 3 - protein, 39 aa, Homo sapiens
  source 1..39: note = Where X is an amino acid residue other than cysteine
KQFTKXELSQLLKDIDGYGGIALPELIXTMFHTSGYDTQ

SEQ ID NO: 4 - protein, 43 aa, Homo sapiens
  source 1..43: note = Where X is an amino acid residue other than cystein
LDDDITDDIMXAKKILDIKGIDYWLAHKALXTEKLEQWLXEKL

(19) United States
(12) Patent Application Publication
(10) Pub. No.: US 2026/0108588 A1
(43) Pub. Date: Apr. 23, 2026

(54) A COMPLEX COMPRISING A ALPHA-LACTALBUMIN AND A FATTY ACID OR LIPID FOR USE IN THE TREATMENT OR PREVENTION OF CANCER

(71) Applicant: LINNANE PHARMA AB, Malmö (SE)

(72) Inventors: Catharina SVANBORG, Lund (SE); Tran Thi HIEN, Lund (SE)

(73) Assignee: LINNANE PHARMA AB, Malmö (SE)

(21) Appl. No.: 19/117,750

(22) PCT Filed: Oct. 3, 2023
(86) PCT No.: PCT/IB2023/059909

§ 371 Date: Apr. 2, 2025

(30) Foreign Application Priority Data
Oct. 3, 2022 (GB) 2214514.8
Dec. 21, 2022 (GB) 2219335.3

(51) Int. Cl. A61K 38/38; A61K 9/00; A61K 31/201; A61P 35/00

(52) U.S. Cl. CPC A61K 38/38; A61K 9/0053; A61K 31/201; A61P 35/00

(57) ABSTRACT

The invention provides a complex for use in various therapeutic applications, and methods of treatment using the complex, or pharmaceutical compositions comprising the complex. The complex is particularly useful for treating malignant transformations, particularly cancer, especially where such transformations are found at a site distant from the site of administration of the complex, as well as for the treatment of metabolic-related conditions. The complex comprises a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof.

### TECHNICAL FIELD

The present invention relates to a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof for use in therapy for tumor surveillance, for the prevention or treatment of cancer and to other conditions, including metabolic-related disorders, whether secondary to cancer or independent thereof.

### BACKGROUND

HAMLET (human alpha-lactalbumin made lethal to tumor cells) is the first member of a family of tumoricidal unfolded protein-lipid complexes, consisting of partially unfolded α-lactalbumin and oleic acid. Initially isolated in the form of a fraction obtained by passing a casein containing fraction of human milk down an ion exchange column under high salt conditions (WO96/004929), it was found to be biologically active and in particular had an antibacterial activity. Subsequently, other methods for preparing active complexes have been derived including methods in which α-lactalbumin from various sources and oleic acid are heated together in solution. In addition, however, HAMLET and related complexes such as BAMLET, derived from bovine alpha-lactalbumin, have been found to kill transformed cells such as tumor cells or papilloma cells, as well as having antiviral activity. HAMLET kills many types of tumor cells in vitro and this tumoricidal activity is maintained in vivo, as shown in animal models of human glioblastoma xenografts and bladder cancer. Topical application of HAMLET removed or reduced skin papillomas and local instillations of HAMLET killed bladder cancer cells but not healthy cells in surrounding tissues and caused a reduction in tumor size. The sensitivity of tumor cells to HAMLET reflects oncogenic transformation and is modified by the glycolytic state of the cell (Storm P, et al. (2011). Oncogene). shRNA silencing of c-Myc or Ras pathway members conferred resistance to HAMLET and the level of c-Myc expression paralleled HAMLET sensitivity. Furthermore, glucose deprivation sensitized tumor cells to HAMLET and the HAMLET-sensitivity was modified by shRNAs targeting glycolytic enzymes. Additionally, HAMLET was shown to have pronounced effects on global metabolism with a rapid metabolic paralysis in tumor cells and potential diversion of the glycolytic flux towards the pentose phosphate pathway.

Tumor surveillance is essential to prevent tumor cells from developing into a tumor mass. The protective forces that remove emergent tumor cells or reprogram them towards health are poorly understood, however. The tissue environment is expected to contain molecules that execute the anti-tumor defense, but even the role of immune surveillance remains unclear, as immunodeficiencies per se do not appear to cause cancer. Tissue development in the newborn presents a similar challenge, as immature cells or viruses infected cells need to be removed and replaced by cells that carry out essential physiological functions in mature tissues. Molecules provided in milk have evolved to provide solutions locally, in the respiratory tract and gastrointestinal tract. Molecular solutions that remove immature cells and drive tissue differentiation, provided in the milk, may therefore be highly relevant also to achieve tumor surveillance therapeutically.

Alpha-lactalbumin is the most abundant protein in human milk and is crucial for the survival or the offspring. Native alpha-lactalbumin acts as a substrate specifier in the lactose synthase complex and without lactose, milk cannot be expressed, due to high viscosity. When partially unfolded, human alpha-lactalbumin gains the ability to kill tumor cells and immature cells, by forming oleic acid complexes. HAMLET (Human alpha-lactalbumin made lethal to tumor cells) effectively kills a wide range of tumor cells and has shown therapeutic efficacy in colon cancer and other several cancer models and clinical studies. A second HAMLET family member, alpha1-oleate, formed by the N-terminal alpha-helical peptide of alpha-lactalbumin, has shown therapeutic efficacy in patients with bladder cancer.

Whilst complexes such as HAMLET have been demonstrated previously as being therapeutic in the treatment of a range of pre-existing cancers (WO2005/082406), and for the prophylactic treatment of colon cancer (WO2014/023976), it has not been shown previously that such complexes are useful in the treatment of cancers to which the complexes cannot be directly applied (e.g., peroral application for cancers outside the GI tract). Surprisingly, the inventors have now demonstrated that the complexes are useful for the treatment of cancers that are remote from the site of administration, and of secondary cancers or metastases. The nature of the complex is such that one would not expect it to be up taken from site of administration. Accordingly, the ability of the complex to act at a site remote from its original administration is highly surprising. Further, the inventors have demonstrated that the complexes have long term effects, lasting beyond the period of administration, allowing the inventors to identify the usefulness of the complexes in the prevention or treatment of secondary or de novo cancers, which is, again, significant and surprising.

The inventors investigated how the bovine alpha-lactalbumin complex BAMLET affects intestinal tumor development and tissue homeostasis, comparing intestinal cancer-prone adenomatous polyposis coli multiple intestinal neoplasia (Apc^{Min/+}) mice to C57BL/6 controls. The results indicate that BAMLET supplementation of the drinking water is sufficient to delay tumor progression and increase the survival of tumor-prone mice. In-depth analysis revealed profound effects on the transcriptional machinery affecting the tumor microenvironment. These effects included the programmed cell death-1 (PD-1) signaling pathway, which was inhibited. Unexpected effects on Wnt/β-catenin signaling in lungs, livers, kidneys were also observed, suggesting a protective effect of BAMLET beyond the intestine. Healthy mice, in contrast, showed a weak intestinal response, affecting metabolic functions such as lipid and glucose metabolism and insulin resistance, with no evidence of systemic effects. The results illustrate how the need for tumor surveillance is met by a milk constituent that preferentially targets tumor cells, in predisposed hosts without detrimental effects in a healthy host background. The response to BAMLET in extra-intestinal tissues further suggested a more general role of alpha-lactalbumin for tissue development in the newborn and in tumor surveillance.

Based on the evidence collected, it is clear that the complexes have a systemic impact, changing the overall tumor environment. As set out below, the inventors have produced evidence of both a shift in genetic expression in response to administration of the complexes and a physiological impact. Further, whereas most cancer therapies are directed at a single point of attack, for example by targeting one specific gene, the complexes of the invention have a remarkably broad efficacy, tackling cancers via multiple routes. This enables the complexes to be useful in targeting many different cancers, including metastases.

The inventors have also identified that administration of the complexes provides long term protection from cancers.

The inventors have further identified other systemic effects, particularly metabolic effects. Such effects have been found in otherwise healthy animals. The effects may impact cancer development and/or the general progress of cancer patients due to the reduction of conditions secondary to cancer that significantly impact health. The effects also demonstrate the usefulness of the complexes in treating such conditions when they are unrelated to cancer.

### SUMMARY OF THE INVENTION

The invention provides a complex for use in various therapeutic applications, and methods of treatment using the complex, or pharmaceutical compositions comprising the complex. The complex is particularly useful for treating malignant transformations, particularly cancer, especially where such transformations are found at a site distant from the site of administration of the complex.

The complex comprises a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof.

There is provided a complex as defined for use in tumor surveillance. Further provided is a complex as defined for use in altering the tumor environment. Also provided is a method of treating or preventing cancer, comprising the step of tumor surveillance and/or the step of altering the tumor environment.

Tumor surveillance means the identification of cancerous or pre-cancerous cells, or other indicators of cancer or a pre-cancerous state. It may also include initiating a response to the presence of such cells or indicators, for example by altering the tumor environment.

Altering the tumor environment means modifying the conditions that affect tumor development or progression, for example, but not limited to, immune cells, signalling molecules, extracellular matrix, blood supply. It can refer to the tumor microenvironment, i.e., the environment surrounding a tumor, or to the broader environment of the body. In particular, altering the tumor environment means modifying the tumor environment such that tumor development, progression or metastasis is reduced or prevented, or the likelihood of tumor development, progression or metastasis is reduced or prevented.

There is provided a complex as defined for use in the prevention or treatment, particularly treatment, of cancer; wherein the complex is for administration at a first site and the cancer is at a second site.

Also provided is a method of preventing or treating cancer, particularly treating, comprising the step of administering an effective amount of the complex or a pharmaceutical composition comprising the complex to a subject having cancer, or a predisposition to, or an increased likelihood of developing a cancer, wherein the complex or composition is for administration to a first site, and the cancer is at a second site.

The complex or a composition comprising the complex is for administration to a first site on or in the body. By this, we mean that the complex is formulated for administration to a particular site, for example it is formulated for peroral, intravesical, intracerebral or topical administration.

The cancer is found at a second site. For example, the cancer is found in one or more of the nasal passage, the GI tract (e.g., in one or more of the oral cavity, the stomach, the colon, the bowel), the brain, the lung, the kidney, the vagina, the bladder, the liver, the skin, the breast, the prostate and/or the ovary.

In particular, the cancer may be found in the lung, the kidney or the liver. Preferably it is found in the lung. Alternatively, it may be found in the kidney. Alternatively, it may be found in the liver.

In some embodiments, the second site is not one or more of the nasal passage, the GI tract (in one or more of the oral cavity, the stomach, the colon, the bowel), the brain, the lung, the kidney, the vagina, the bladder, the liver, the skin, the breast, the prostate and/or the ovary. In particularly, in some embodiments it is not the nasal passage. In some embodiments it is not the GI tract. In some embodiments it is not the brain. In some embodiments it is not the lung. In some embodiments it is not the kidney. In some embodiments it is not the vagina. In some embodiments it is not the bladder. In some embodiments it is not the liver. In some embodiments it is not the skin. In some embodiments it is not the breast. In some embodiments it is not the prostate. In some embodiments it is not the ovary.

The first and second sites are preferably different and, more preferably are remote from one another, i.e., are found in different parts of the body or in different systems. For example, where the complex is for administration perorally, the cancer is not a cancer of the GI tract. Table 1 provides further examples of the first and second sites.

TABLE 1
Routes of administration and cancer to be treated.

| | | Second site (location of cancer) | | | | | | | | | | | |
|---|---|---|---|---|---|---|---|---|---|---|---|---|---|
| | | Nasal | GI | | | | | | | | | | |
| | | passage | tract | Brain | Lung | Kidney | Vagina | Bladder | Liver | Skin | Breast | Prostate | Ovary |
| First site | Intranasal | | X | X | | X | X | X | X | X | X | X | X |
| (route of | Peroral | X | | X | X | X | X | X | X | X | X | X | X |
| administration) | (administration | | | | | | | | | | | | |
| | to the GI tract) | | | | | | | | | | | | |
| | Intracerebral | X | X | | X | X | X | X | X | X | X | X | X |
| | Inhalation | | X | X | | X | X | X | X | X | X | X | X |
| | Perfusion to | X | X | X | X | | X | X | X | X | X | X | X |
| | kidney | | | | | | | | | | | | |
| | Intravaginal | X | X | X | X | X | | X | X | X | X | X | X |
| | Intravesical | X | X | X | X | X | X | | X | X | X | X | X |
| | Perfusion to | X | X | X | X | X | X | X | | X | X | X | X |
| | liver | | | | | | | | | | | | |
| | Topical (to | X | X | X | X | X | X | X | X | | X | X | X |
| | skin) | | | | | | | | | | | | |
| | Intraductal | X | X | X | X | X | X | X | X | X | | X | X |
| | Perfusion to | X | X | X | X | X | X | X | X | X | X | | X |
| | prostate | | | | | | | | | | | | |
| | Direct to ovary | X | X | X | X | X | X | X | X | X | X | X | |
| | Intravenous | X | X | X | X | X | X | X | X | X | X | X | X |

The treatment or prevention of cancer may comprise the step of tumor surveillance and/or altering the tumor environment.

Also provided is a complex as described previously, for use as a checkpoint inhibitor, particularly an inhibitor of PD-1. The invention provides the complex for use in the prevention or treatment, particularly treatment, of PD-L1 positive cancers or other cancers that are susceptible to PD-1 targeting.

The invention further provides a method of preventing or treating a cancer that is PD-L1 positive, or is otherwise susceptible to PD-1 targeting, comprising administering a therapeutically effective amount of the complex, or a composition comprising the complex, to a subject in need thereof.

The cancer may be found at any site in the body, for example at any of the sites listed in relation to the other aspects of the invention.

The complex may be for administration via any suitable route, such as those described in relation to other aspects of the invention. It may be for administration directly to the site of the cancer, or for administration at a different site. For example, the complex may be for administration perorally for the treatment of PD-L1 positive cancers in the GI tract, or elsewhere in the body, such as the liver, lung or kidney.

The cancer may be a primary cancer or a metastasis.

Also provided by the invention is a complex as previously described, for use in the prevention, reduction, or treatment of metastasis.

The invention further provides a method of preventing or treating metastatic cancer, comprising administering a therapeutically effective amount of the complex, or a composition comprising the complex, to a subject in need thereof.

The primary tumor from which the metastasis arises, or the metastasis itself may be found at any site in the body, for example at any of the sites listed in relation to the other aspects of the invention.

The complex may be for administration via any suitable route, such as those described in relation to other aspects of the invention. It may be for administration directly to the site of the primary cancer or the metastasis, or for administration at a different site. For example, the complex may be for administration perorally for the prevention, reduction or treatment of metastatic cancers in, or arising from cancers in the GI tract. Or it may be for prevention, reduction or treatment of metastatic cancers, or arising from cancers, elsewhere in the body, such as the liver, lung or kidney.

The prevention, reduction, or treatment of metastasis may comprise the step of tumor surveillance and/or altering the tumor environment.

Also provided by the invention is a complex as previously described, for use in the treatment or prevention of metabolic-related conditions, such as insulin resistance, type II diabetes, metabolic syndrome, non-alcoholic fatty acid liver disease, cirrhosis, high blood pressure. The complex may be used to modulate insulin tolerance or sensitivity, lipid metabolism and/or glucose metabolism and is therefore useful in the treatment of conditions arising from challenges with such processes.

The invention further provides a method of treating such metabolic-related conditions, comprising administering a complex as defined to a subject.

Such metabolic conditions may be related to the presence of cancer, for example they may be secondary to cancer, or may be independent thereof. The complex is particularly useful for improving the health of a subject having cancer, by treating the cancer, or treating conditions secondary to the cancer, or both. Accordingly, the invention provides a complex as previously described, for use in the treatment or prevention of metabolic-related conditions, such as insulin resistance, type II diabetes, metabolic syndrome, non-alcoholic fatty acid liver disease, cirrhosis, high blood pressure, in a subject that has, or has previously had, cancer. Also provided is a complex as previously described, for use in the treatment or prevention of metabolic-related conditions, such as insulin resistance, type II diabetes, metabolic syndrome, non-alcoholic fatty acid liver disease, cirrhosis, high blood pressure, in a subject that does not have, or has not had cancer.

### The Complex

The complex comprises a polypeptide having a sequence of a naturally occurring alpha-lactalbumin, or a functional variant thereof; or a peptide of up to 50 amino acids comprising an alpha-helical domain of said polypeptide, and a fatty acid or lipid or salt thereof.

In one embodiment, the polypeptide has a sequence of a naturally occurring alpha-lactalbumin, preferably a human or bovine alpha-lactalbumin, more preferably a bovine alpha-lactalbumin.

In one embodiment, the alpha-helical domain is the Alpha 1 (residues 1-39) or Alpha 2 (residues 81-123) domain of human alpha-lactalbumin, being of SEQ ID NO 3 or SEQ ID NO 4; KQFTK XELSQLLKDIDGYGGIALPELI XTMFHTSGYDTQ (SEQ ID NO 3) LDDDITDDIM XAKKILDIKGIDYWLAHKALXTEKLEQWL XEKL (SEQ ID NO 4) where X is an amino acid residue other than cysteine.

In an embodiment, the complex comprises a peptide of about or less than 45, 42, or 40 amino acids, in particular 39 amino acids, preferably corresponding to the Alpha 1 domain of human alpha-lactalbumin.

In one embodiment, the functional variant consists of a sequence lacking disulfide bonds. In one embodiment, the functional variant consists of a sequence in which cysteine residues in the native alpha-lactalbumin are changed to other amino acid residues, preferably alanine residues.

In one embodiment, the fatty acid or lipid or salt thereof is a fatty acid or salt thereof. In one embodiment, the fatty acid or salt thereof is oleic acid or an oleate salt.

In one embodiment, the polypeptide has the sequence of bovine alpha-lactalbumin and the fatty acid or salt thereof is oleic acid or an oleate salt.

The polypeptide present in the complex may have the sequence of an α-lactalbumin or a variant thereof as described above.

The complex may be referred to as a biologically active complex. As used herein, the term “biologically active” means that the complex has a biological activity, which is different from, or stronger than the individual components. In particular, the complex is able to induce cell death in particular selectively in tumor cells and/or has a bactericidal or antiviral effect not seen with the native protein including for example monomeric α-lactalbumin forms, although other therapeutic effects may be available.

The expression “variant” refers to proteins or polypeptides having a similar biological function but in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt the function of the DNA binding domain polypeptides. Broadly speaking, fewer non-conservative substitutions will be possible without altering the biological activity of the polypeptides.

Determination of the effect of any substitution (and, indeed, of any amino acid deletion or insertion) is wholly within the routine capabilities of the skilled person, who can readily determine whether a variant polypeptide retains the fundamental properties and activity of the basic protein. For example, when determining whether a variant of the polypeptide falls within the scope of the invention, the skilled person will determine whether complexes comprising the variant retain biological activity (e.g., tumor cell death) of complexes formed with unfolded forms of the native protein and the polypeptide has at least 60%, preferably at least 70%, more preferably at least 80%, yet more preferably 90%, 95%, 96%, 97%, 98%, 99% or 100% of the native protein.

Variants of the polypeptide may comprise or consist essentially of an amino acid sequence with at least 70% identity, for example at least 75%, 80%, 85%, 90%, 91%, 92%, 93%, 94%, 96%, 97%, 98% or 99% identity to a native protein sequence such as an alpha-lactalbumin or lysozyme sequence.

The level of sequence identity is suitably determined using the BLASTP computer program with the native protein sequences as the base sequence. This means that native protein sequences form the sequence against which the percentage identity is determined. The BLAST software is publicly available at http://blast.ncbi.nlm.nih.gov/Blast.cgi (accessible on 12 Mar. 2009).

In a particular embodiment, the polypeptide is an α-lactalbumin such as human, bovine or ovine α-lactalbumin. Whilst variants of these as described above may be useful in the invention, for nutraceutical use in particular, it may be preferable to utilize the native proteins in the products. A particular embodiment used human α-lactalbumin. In another embodiment, the α-lactalbumin is bovine α-lactalbumin. The sequence of a wide range of α-lactalbumins is known in the literature, for example as shown in Watanabe et al., J. Vet Med Sci, (2000) 62(11); 1217-1219.

In another embodiment, the polypeptide comprises a recombinant protein having the sequence of α-lactalbumin or a fragment thereof but which lacks intra-molecular disulfide bonds or cross-links. By ensuring that the recombinant protein lacks intra-molecular disulfide crosslinks, the molecule will be three-dimensionally non-native and completely inactive in terms of its original endogenous biological activity. This is achieved by changing cysteine residues in the native α-lactalbumin to other residues, in particular alanine residues. Preferably all cysteine residues will be changed to other residues, such as alanine residues. In particular the recombinant protein is based upon the sequence of human α-lactalbumin but α-lactalbumin from other sources, including bovine or ovine α-lactalbumin may be used to derive the recombinant protein.

In a particular embodiment, the polypeptide is a recombinant protein having the sequence of native mature α-lactalbumin but which has all of the cysteines found at positions 6, 28, 61, 73, 77, 91, 111 and 120 in the full length sequence of mature human α-lactalbumin mutated to other amino acids, such as alanine, which do not give rise to disulphide bridges. Thus, a particular of a protein that may be utilised in accordance with the invention comprises a protein of SEQ ID NO 1.

(SEQ ID NO 1)
KQFTKAELSQLLKDIDGYGGIALPELIATMFHTSGYDTQAIVENNESTEY
GLFQISNKLWAKSSQVPQSRNIADISADKFLDDDITDDIMAAKKILDIKG
IDYWLAHKALATEKLEQWLAEKL

where the bold type indicates positions of mutations of cysteines in native human α-lactalbumin.

As reported in WO2010079362, additional amino acid residues, for example up to 20 amino acids, may be attached at N and/or C terminal of the protein, if convenient, for example for expression purposes. Thus in particular, a recombinant protein as shown in SEQ ID NO. 1 but with an additional methionine at the N-terminus (SEQ ID NO 2 shown below) has been used in the complex of the invention.

(SEQ ID NO 2)
MKQFTKAELSQLLKDIDGYGGIALPELIATMFHTSGYDTQAIVENNESTE
YGLFQISNKLWAKSSQVPQSRNIADISADKFLDDDITDDIMAAKKILDIK
GIDYWLAHKALATEKLEQWLAEKL

The polypeptide used in the complex is suitably in pure form, and is suitably prepared using conventional methods of peptide synthesis or by recombinant expression. In particular, DNA encoding the required recombinant α-lactalbumin can be inserted into suitable expression vectors such as plasmids, which can then be employed to transform host cells, for example, prokaryotic cells such as E. coli or eukaryotic cells such as particular insect cells using conventional methods.

Suitable fatty acids or lipids include those known to provide biologically active complexes. These include fatty acids, for example as described in WO2008058547. Where salts are used, these are suitably water soluble salt. Particular examples of suitable salts may include alkali or alkaline earth metal salts. In a particular embodiment, the salt is an alkali metal salt such as a sodium- or potassium salt. Where used in pharmaceuticals, the salts will be pharmaceutically acceptable.

Particular examples of fatty acids or lipids used in the present invention are those having from 4-30, for example from 6 to 28, such as from 8 to 26 carbon atoms. In particular embodiments, the fatty acid or lipid has from 10 to 24, such as from 12 to 22, for example from 14 to 20 carbon atoms. In particular, the fatty acid or lipid will have 16, 17, 18 or 20 carbon atoms. The fatty acids may be saturated or unsaturated.

In particular however, the complexes of the invention utilize fatty acids or salts of fatty acids having 18 carbon atoms. In one embodiment, the complexes of the invention utilize fatty acids or salts of fatty acids having 18 carbon atoms and wherein the fatty acid chain is unsaturated. In one embodiment, the fatty acid or salt of the fatty acid is a C18:1 fatty acid or salt thereof. A specific example is a C18:1 fatty acid or salt thereof of formula CH₃(CH₂)₇CH═CH(CH₂)₇COOH or CH₃(CH₂)₇CH═CH(CH₂)₇COO⁻. In one embodiment, the fatty acid or salt thereof is oleic acid or oleate salt.

The complex may be prepared using methods similar to those described for example in WO99/26979, WO2008/138348, WO2010/131237, WO2014/023976, WO2018/210759, and WO2022/073982 the content of which is incorporated herein by reference. Not only has it been found that complexes can be prepared by contacting unfolded α-lactalbumin or derivatives thereof with co-factors in particular oleic acid or salts thereof under ion exchange conditions such as those found on an ion exchange column, but also incubation of solutions of α-lactalbumin or derivatives thereof with a co-factor at elevated temperatures, for example of from 50-80° C., for example from 50-70° C. and in particular between 55-60° C. will result in the production of suitable complexes for use in the invention.

These methods however have generally focused on attempting to recreate the conditions in which the protein becomes unfolded and complexed with oleic ions. Such work has focused on using pure proteins including recombinant variant versions of the base proteins to facilitate the production of active complexes. Such starting materials however can also increase the cost of production.

It is known that complexes obtained using α-lactalbumin from sources other than human milk, and in particular, BAMLET, obtained using bovine α-lactalbumin shows a qualitatively similar effects on cells and in particular on tumor cells as HAMLET (see for instance, Rammer et al. (2010) Mol. Cancer Ther. 9(1) 24-32). Therefore, effects demonstrated hereinafter using BAMLET would be similarly observed if HAMLET or compositions based upon HAMLET are used instead of BAMLET.

### Dosage

The amount of complex administered to an individual will depend upon a variety of factors including the nature of the composition as well as the risk factor. However, as a general rule, when administered perorally, from 1 mg to 20 g/dose of the biologically active complex is used for each administration, which is suitably administered daily. The daily dose may be, for example, at least or about 1 mg, 2 mg, 5 mg, 10 mg, 15 mg, 20 mg, 25 mg, 50 mg, 75 mg, 100 mg, 200 mg, 300 mg, 400 mg, 500 mg, 750 mg, 1 g, 2 g, 3 g, 4 g, 5 g, 7.5 g, 10 g, 12.5 g, 15 g, or 17.5 g. Alternatively, or additionally, the daily dose may be less than 25 g, 22.5 g, 20 g, 17.5 g, 15 g, 10 g, 7.5 g, 5 g, 4 g, 3 g, 2 g, 1 g, 750 mg, 500 mg, 400 mg, 300 mg, 200 mg, 100 mg, 75 mg, 50 mg, 25 mg, 20 mg, 15 mg, 10 mg or 5 mg. Alternatively, the complex may be for administration in a dosage of 0.1 g to 1 g per kg of bodyweight, daily. It may be for administration in a dosage of at least or about 0.1 g, 0.2 g, 0.3 g, 0.4 g, 0.5 g, 0.6 g, 0.7 g, 0.8 g, 0.9 g or 1 g per kg of bodyweight, daily. Alternatively, or additionally, it may be for administration in a dosage of less than 1.5 g, 1 g, 0.9 g, 0.8 g, 0.7 g, 0.6 g 0.5 g, 0.4 g, 0.3 g, 0.2 g, 0.1 per kg of bodyweight, daily.

### Food and Beverage Compositions

The complex or pharmaceutical composition may be in the form of a beverage, particularly drinking water, or a foodstuff, such as baby-food, or as an additive or component for a beverage or foodstuff, such as a powder for mixing into a drink, for example, in the manner of a protein shake. Such food and beverage compositions may be produced using standard techniques. Similarly, the complex or pharmaceutical composition may be in the form of a composition for providing parental or, preferably, intravenous nutrition.

The invention also provides a foodstuff, beverage, food additive or other nutritional composition comprising the complex as defined, particularly for use in treating cancer or a metabolic-related condition, as described in earlier aspects of the invention.

Examples of such compositions include water-based or milk-based drinks, particularly drinking water; baby-food; nutritional compositions for parental or intravenous administration; food additives, for example powders for mixing into drinks or food; nutritional capsules, gels or tablets.

### General

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows that BAMLET treatment delays tumor progression in Apc^{Min/+} mice, wherein: a, Schematic representations of the treatment model. Ten-week-old female Apc^{Min/+} mice received daily 20 mg of BAMLET or PBS (sham) by gavage, twice daily for ten days. The mice were sacrificed two weeks (PBS: n=4+5, BAMLET: n=5+5) or five weeks (PBS: n=5+5, BAMLET: n=5+5) after the end of treatment (2w or 5w post-treatment, pt). b, Ten-week-old female mice received daily 20 mg of BAMLET or PBS in the drinking water (dw) until sacrifice after eight weeks (PBS: n=5+5+5, BAMLET: n=5+5+9) or were followed long term (8w drinking water, dw) (PBS: n=5+8, BAMLET: n=6+9). c, H&E stained intestinal sections showing the progression of colon cancer in the sham group from microadenomas (2w pt) to polyps (5w pt) and larger tumors (8w dw). Representative sections, n=4-5 mice per group. d, BAMLET prevented or delayed the progression of colon cancer at each time point. e, The total number of polyps was significantly reduced in BAMLET treated Apc^{Min/+} mice and f, the tumor areas were smaller compared to the sham group. Data are presented as means±S.E.M. of two to three experiments (n=9-15 mice for sham group, n=10-19 mice for BAMLET treated group).

FIG. 2 shows potent effects of BAMLET on intestinal gene expression, wherein: a, Heat map comparing intestinal gene expression profiles between the BAMLET-treated Apc^{Min/+} mice and the Apc^{Min/+} sham mice. A time-dependent increase in the number of regulated genes was observed in treated mice receiving BAMLET by gavage (two or five weeks post treatment, 2w or 5w pt) and the effect was confirmed in mice receiving BAMLET in the drinking water (eight weeks, 8w dw). (Red: upregulated genes, blue: downregulated genes, black: not regulated genes, cut-off fold change ≥2.0 compared to sham). b, Venn diagram of significantly regulated genes in BAMLET treated Apc^{Min/+} mice identifying genes regulated at all time points. c, Network analysis of these shared genes (n=2339) revealed major treatment effects. Wnt/β-catenin signaling was inhibited in BAMLET treated Apc^{Min/+} mice. d, Genes defining the tumor microenvironment were broadly inhibited, predicted to reduce proliferation, angiogenesis, metastasis, and the PD-1 pathway. e, Biofunctions such as tumor growth, cell movement, invasion and metastasis were inhibited. f, Tumor microenvironment genes were also significantly regulated.

FIG. 3 shows long-term effects of BAMLET in the drinking water, wherein a, Schematic representation of the long-term treatment model. Apc^{Min/+} mice received drinking water (dw) supplemented with BAMLET (20 mg daily) (n=6+9) or PBS (sham) (n=5+8) from ten weeks of age. b, c, BAMLET treatment increased survival, compared to sham treated mice. d, Reduction in polyp number and e, body weight loss in BAMLET treated compared to sham treated Apc^{Min/+} mice. f, Gene expression analysis identified the Wnt/β-catenin signaling pathway as activated in sham treated Apc^{Min/+} mice (15 weeks PBS in drinking water, 15w dw) but inhibited or not regulated in BAMLET treated Apc^{Min/+} mice (27 weeks BAMLET in drinking water, 27w dw). g, Reduction of β-catenin staining in intestinal sections of BAMLET treated mice compared to sham treated Apc^{Min/+} mice. h, Quantification of β-catenin in intestinal, lung, liver and kidney tissues section. Data are presented as means±S.E.M. of two experiments (n=5+8 mice for sham group, n=6+9 mice for BAMLET treated group).

FIG. 4 shows inhibition of PD-1 signaling by BAMLET supplementation in the drinking water, wherein: a, Gene expression analysis of intestinal RNA identified the PD-1 pathway as strongly up-regulated in sham treated APC^{Min/+} mice compared to healthy C57BL/6 mice at long-term follow up. b, Genes in the PD-1 pathway were not regulated in BAMLET treated APC^{Min/+} mice compared to healthy C57BL/6 mice (cut off FC 2, p<0.05). c, Genes in the PD-1 pathway were inhibited in BAMLET treated compared to sham treated APC^{Min/+} mice. d,e, PD-1 staining of intestinal sections from sham treated or BAMLET treated APC^{Min/+} mice were stained with anti-PD-1 antibodies (red=PD-1, blue=DAPI). d, Swiss roll preparation of intestinal segments from sham or BAMLET treated mice. Arrows indicate the position of the tumor area magnified in e. Representative images, n=3 mice per group. f, Quantification of PD-1 staining in the Swiss roll preparation, comparing tumor and healthy areas of sham treated and BAMLET treated APC^{Min/+} mice. Data are presented as means±S.E.M. of n=3 mice per group. g, h, Quantification of PD-1 staining in intestinal sections of individual mice comparing tumor areas to healthy areas.

FIG. 5 shows inhibition of lung cancer by BAMLET long term treatment, wherein a, Intestinal sections from BAMLET treated or sham treated APC^{Min/+} mice were examined by immunohistochemistry, after staining with β-catenin- or TTF-1-specific antibodies followed by H&E counterstaining. a,b,c, β-catenin staining and d,e,f, TTF-1 staining in healthy C57/BL6 mice, sham treated- or BAMLET treated APC^{Min/+} mice. a C57/BL6 mice showed a normal pattern of weak β-catenin staining in bronchial and bronchiolar epithelial cells. b, Significant increase in overall β-catenin staining in sham treated APC^{Min/+} mice, including foci adjacent to the bronchiolar epithelium with enhanced staining. c, Reduced β-catenin in BAMLET treated compared to sham treated APC^{Min/+} mice. d, Quantification of β-catenin staining in lung tissues for (a-c) n=3 mice per group. e, C57/BL6 mice showed weak TTF-1 staining in bronchial and bronchiolar epithelial cells. f, Significant increase in overall TTF-1 staining in sham treated APC^{Min/+} mice, including areas of proliferating cells with enhanced staining emanating from the bronchiolar epithelium. g, Reduced TTF-1 staining in BAMLET treated APC^{Min/+} mice. h, Quantification of TTF-1 positive areas in whole lung sections (see inset). Data are presented as means±S.E.M. of n=3 mice per group.

FIG. 6 shows effects of BAMLET in healthy C57BL/6 mice, wherein BAMLET was administered to healthy C57BL/6 mice by gavage (two weeks and five weeks) (n=5) or in drinking water (eight weeks) (n=5). Intestinal tissue was subjected to gene expression analysis. a, Heat map comparing gene expression profiles mice receiving BAMLET by gavage and followed for two or five weeks (2w or 5w pt) or BAMLET supplemented drinking water for eight weeks (8w dw) (Red: upregulated genes, blue: downregulated genes, black: not regulated genes, cut-off fold change ≥1.5, P<0.05, compared to sham). b, The total number of regulated genes was low in the healthy C57BL/6 mice (about 150 genes) with no evidence of a toxic response to BAMLET. c, Venn diagram of significantly regulated genes in the BAMLET treated C57BL/6 mice. d, Biofunction analysis of the common genes identified in (c) predicted effects on lipid metabolism, glucose metabolism, insulin tolerance and inflammation. e, P values and Z scores of biofunctions regulated in BAMLET treated C57BL/6 mice after five weeks. f, Top regulated common genes were mostly enzymes related to carbohydrate, lipid and protein digestion. g, Schematic of the BAMLET effects discussed in this study.

FIG. 7 shows supplementary data for FIG. 1, showing BAMLET treatment delays tumor progression in Apc^{Min/+} mice, wherein a-e, Data from Apc^{Min/+} mice treated by oral gavage and followed for two weeks post treatment (2w pt) (n=5+5 for BAMLET; n=4+5 for sham). f-j, Data from Apc^{Min/+} mice treated by oral gavage and followed for five weeks (5w pt) (n=5+10 for BAMLET; n=5+5 for sham). a, f, Dissection photomicrographs of small intestinal segments showing tumors (arrowheads) in BAMLET treated or sham treated Apc^{Min/+} mice. b, g, Methylene blue-stained whole mounts of the dissected intestinal segments (arrowheads, tumors), n=4 mice per group. c, h, The total number of polyps was significantly reduced two and five weeks pt in BAMLET treated compared to sham treated Apc^{Min/+} mice. d, i, The polyp number and size of detected polyps was reduced by BAMLET treatment (<0.5 mm, 0.5-2 mm, >2 mm). e, j, H&E stained intestinal Swiss roll sections showing smaller and fewer polyps in BAMLET treated Apc^{Min/+} mice than in sham treated mice, n=4 mice per group. Data are presented as means±S.E.M. of two to three experiments (n=9-15 mice for sham group, n=10-19 mice for BAMLET treated group).

FIG. 8 shows BAMLET administration into the drinking water, eight weeks follow up, wherein Apc^{Min/+} mice received BAMLET-supplemented drinking water or PBS for eight weeks (8w dw). a, Dissection photomicrographs of small intestinal segments showing tumors (arrowheads) in BAMLET treated or sham treated Apc^{Min/+} mice after eight weeks. b, Total number of polyps was significantly reduced in BAMLET treated intestine compared to sham and c, the polyps number reduction reflected all the three sizes analyzed (<0.5 mm, 0.5-2 mm, >2 mm). Data are presented as means±S.E.M. of two experiments (n=5+8 mice for sham treated Apc^{Min/+} mice group, n=6+9 mice for BAMLET treated Apc^{Min/+} mice group). d, Methylene blue stained whole mounts of intestinal segments (arrowheads, tumors), n=4 mice per group. e, H&E-stained intestinal Swiss roll sections showing smaller and fewer polyps in BAMLET treated Apc^{Min/+} mice than in sham after eight weeks of treatment, n=4 mice per group. f, Heat map comparing gene expression profiles between the sham treated APC^{Min/+} mice and mice receiving BAMLET in the drinking water. (Red: upregulated genes, blue: downregulated genes, black: not regulated genes, cut-off fold change ≥2.0 compared to healthy intestinal tissue). g, Histograms showing the number of regulated genes. h, Biofunction analysis of the regulated genes showed a remarkable reduction in cancer functions in the BAMLET treated group compared to sham.

FIG. 9 shows effects of BAMLET treatment on intestinal gene expression, wherein a, d, Heat maps comparing gene expression profiles between the sham treated Apc^{Min/+} mice and mice receiving BAMLET by gavage. Mice were sacrificed two weeks (upper panel) or five weeks (lower panel) after the end of treatment (2w or 5w pt). (Red: upregulated genes, blue: downregulated genes, black: not regulated genes, cut-off fold change ≥2.0 compared to healthy intestinal tissue). b, e, Histograms showing the number of regulated genes. Upper panel: 2w pt; Lower panel: 5w pt. Gene expression was increased in sham treated mice after two weeks compared to healthy mice and a further increase was observed after five weeks. In contrast, the number of regulated genes was markedly reduced in BAMLET treated Apc^{Min/+} mice after two weeks with a further reduction after five weeks. c, f, Biofunction analysis revealed a remarkable reduction in cancer-related functions in all treatment groups compared to sham. g, Top regulated genes in the sham- and BAMLET treated Apc^{Min/+} mice. Cancer related biofunctions predominated in the sham group but were inhibited in the BAMLET-treated Apc^{Min/+} group. h, Principal component analysis of mRNA profiles in whole intestinal tissue. Mice treated with BAMLET formed a cluster near healthy mice and distant from the sham group. i, A time-dependent reduction in the molecular mechanisms of cancer pathway genes was observed.

FIG. 10 shows inhibition of colon cancer related gene expression by BAMLET treatment, wherein a, Heat maps showing a reduction in the number of colon cancer related genes in Apc^{Min/+} mice receiving BAMLET by gavage (two or five weeks post treatment, 2w or 5w pt) and in the drinking water (eight weeks, 8w dw) compared to sham treated Apc^{Min/+} mice. (Red: upregulated genes, blue: downregulated genes, black: not regulated genes, cut-off fold change ≥2.0 compared to healthy intestinal tissue). b, Histograms showing the number of colon cancer related genes. c, Top regulated colon cancer genes identified by biofunction analysis.

FIG. 11 shows effects of BAMLET treatment on tumor markers by immunohistochemistry of intestinal sections. The levels of tumor markers VEGF, Ki67, Cyclin D1 and β-catenin were reduced in BAMLET treated Apc^{Min/+} mice compared to sham treated Apc^{Min/+} mice. a, Five weeks post oral gavage quantified in (b). c, Eight weeks of BAMLET supplemented drinking water, quantified in (d). Data are presented as means±S.E.M., n=5 mice per group.

FIG. 12 shows effects of BAMLET on colorectal carcinoma cells, wherein a-b, Live-cell confocal images showing uptake by DLD1 colorectal carcinoma cells of Alexa Fluor-568 labelled BAMLET (21 μM, Magenta). Nuclei are counterstained with DAPI (blue). b, Quantification of uptake in a. c, Membrane response to BAMLET in giant unilamellar vesicles (GUVs, magenta). d, Quantification of the response in c. e, Dose-dependent increase in TUNEL staining in BAMLET treated DLD1 colorectal adenocarcinoma cells (n=50 cells per group). Scale bar=20 μm. f, Quantification of TUNEL staining. g, Tumor cell death quantified by Prestoblue and ATPlite™. BAMLET (orange) was compared to alpha1-oleate (blue) in DLD1 cells, h, Tumor cell survival, defined by the colony assay in human colorectal carcinoma DLD1 and HT29 cells. BAMLET (orange) was compared to alpha1-oleate (blue) Effects of BAMLET and alpha1-oleate on i, Quantifications of the colony assay in (h). j, In vivo imaging of tumor bearing APC^{Min/+} mice or C57BL/6 mice (both 18 weeks old) exposed by gavage to VivoTag 680-labeled BAMLET. The retention of BAMLET was higher after 24 hours (n=3) and 48 hours (n=3) compared to C57BL/6 mice (n=4+4). k, Quantification of the fluorescence intensity in intestinal sections, 24 hours (upper) and 48 hours (lower) after BAMLET administration. l, BAMLET staining in the tumor area of intestinal sections from (k), using immunohistochemistry. m, Quantification of the BAMLET staining from (l), Data are presented as means±S.E.M. from three independent experiments for all cell culture experiments or n=3-4 mice per group.

FIG. 13 shows supplementary data for FIG. 3, showing gene expression analysis of intestinal tissues from BAMLET treated Apc^{Min/+} mice compared to sham treated Apc^{Min/+} mice, wherein a-d, Molecular mechanisms of cancer. colorectal cancer metastasis, tumor microenvironment and Wnt/β-catenin signaling pathways were down-regulated in the BAMLET treated group.

FIG. 14 shows supplementary data for FIG. 3, showing effects of long-term treatment on major tissues outside the intestinal tract, wherein a, Macroscopic appearance of the lungs, livers, kidneys and spleens obtained sham treated (15 weeks, dw) and BAMLET treated (27 weeks, dw) APC^{Min/+} mice compared to healthy C57BL/6 mice at sacrifice after long-term follow up. Changes in tissue morphology indicated systemic involvement in the sham group. These effects were reduced in the group receiving BAMLET supplemented drinking water. b, Tissue analysis in H&E stained sections, n=4 mice per group. c, Gene expression analysis of lungs, livers, kidneys and spleens from BAMLET treated and the sham treated Apc^{Min/+} mice. Heat map comparing gene expression profiles (Red: upregulated genes, blue: downregulated genes, cut-off fold change ≥2.0 compared to sham). d, Total number of regulated genes in lung, liver, kidney and spleen tissues of BAMLET treated mice compared to sham (cut-off fold change ≥2.0 compared to sham). e, The molecular mechanism of cancer pathway was strongly regulated by BAMLET treatment, as well as the colorectal cancer metastasis, tumor microenvironment and Wnt signaling pathway.

FIG. 15 shows effects of BAMLET on systemic β-catenin staining, wherein β-catenin staining was quantified in tissue sections from the liver and kidney tissues of sham treated and BAMLET treated APC^{Min/+} mice and compared to healthy C57BL/6 controls. Representative sections, n=3 mice per group. a-b, Decreased levels of β-catenin staining in liver and kidney tissues from APC^{Min/+} mice treated with BAMLET-supplemented drinking water long term consistent with the inhibition of Wnt/β-catenin signaling outside of the intestinal compartment.

FIG. 16 shows supplementary data for FIG. 3, showing gene expression analysis of lunga, livers and kidneys from BAMLET treated compared to sham treated Apc^{Min/+} mice, wherein the Wnt/β-catenin signaling pathway was strongly up-regulated in lungs, livers and kidneys from sham treated Apc^{Min/+} mice but down-regulated in BAMLET treated Apc^{Min/+} mice.

### DETAILED DESCRIPTION

Targeting early, locally growing tumors can reduce the risk for tumor progression and metastatic disease. While surgery is highly efficient and may remove the tumor permanently, chemotherapy is often required, depending on the tumor classification, predicted risk and spread to lymph nodes prior to surgery. Chemotherapeutic drugs also target healthy tissues; however, the treatment benefits have to be weighed against the risks for toxicity and associated morbidity.

Colorectal cancer is a leading cause of death and >180,000 cases are diagnosed annually in the US¹. Genetic predisposition is a risk factor and mutations affecting the APC gene that may cause both classic and attenuated familial adenomatous polyposis². The APC gene and Wnt/β-catenin signaling network regulate intestinal cell growth and physiology and loss of function mutations may result in cell overgrowth and polyp formation³. Patients with APC mutations may develop large numbers of tumors in the colon in the first few decades of life⁴, and intestinal tumors from Apc mutant mice show similar dynamics⁵. In addition, several lifestyle-related factors have been linked to colorectal cancer, including diet, lack of exercise, smoking and alcohol abuse⁶⁻⁹.

As disclosed herein, the BAMLET complex (bovine alpha-lactalbumin made lethal to tumor cells) was examined as a peroral therapeutic in the Apc^{Min/+} model of intestinal cancer. BAMLET is a complex formed by partially unfolded bovine alpha-lactalbumin and oleic acid and belongs to a new class of tumoricidal molecules, with documented cancer specificity¹⁰⁻¹³. In this study, intestinal polyp formation was inhibited by ten days of BAMLET gavage and long-term protection was achieved by administration of BAMLET into the drinking water. However, surprisingly, the development of systemic disease was prevented by BAMLET treatment and in C57BL/6 mice, which did not develop tumors, health benefits of peroral BAMLET treatment were detected, including effects on lipid metabolism, glycolysis and insulin resistance. These positive effects and the apparent lack of toxicity opens up the possibility for local administration of BAMLET to prevent or treat intestinal cancers and their systemic effects.

This study examined the potential of BAMLET as a peroral therapeutic tool against intestinal cancer. BAMLET was retained in tumor tissue for at least 48 hours after the first oral dose. Ten days of gavage treatment were sufficient to inhibit tumor development and by supplementing BAMLET in the drinking water for eight weeks, tumor development was prevented, and tumor gene expression was inhibited, resulting in a near healthy phenotype. Prolonged treatment delayed tumor development long term and increased the survival of Apc^{Min/+} mice. Remarkably, long-term BAMLET treatment inhibited the PD-1 signaling pathway and prevented systemic disease progression affecting the lungs, liver, kidneys and spleen. These convincing therapeutic effects in Apc^{Min/+} mice suggest that the therapeutic and prophylactic potential of BAMLET should be further explored.

The molecular basis of these therapeutic effects was analyzed by gene expression analysis. Genes that define the tumor microenvironment were strongly upregulated in the sham treated Apc^{Min/+} mice but absent or weakly regulated in the BAMLET group at several early time points. This included genes driving metastasis, tumor growth, angiogenesis and the Wnt/β-catenin signaling pathway. While these effects may reflect the delay in tumor development in treated mice, the observations suggest that BAMLET actively protects the tissue environment by inhibiting or preventing major, cancer-related gene networks from being expressed. Some of these effects were still detected after long-term follow up, suggesting that BAMLET administration into the drinking water maintains an anti-tumor pressure by removing emergent cancer cells and reprogramming gene expression in intestinal tissues.

Unexpectedly, the PD-1 signaling pathway was strongly inhibited by BAMLET, long term. PD-1 and its ligand Programmed Cell Death Ligand 1 (PD-L1) are immunotherapeutic targets, with validated effects in several clinical trials of colorectal, lung, renal cell carcinoma and breast cancers¹⁶⁻²³. The immune checkpoint therapy blocks the PD-1/PD-L1 interaction by directly targeting tumor cells or indirectly enhancing or restoring T cell function and thus anti-tumor activity^{24,25}. BAMLET treated Apc^{Min/+} mice showed reduced PD-1 pathway activation compared to the sham group, where the intestinal PD-1 signaling pathway was upregulated and PD-1 staining was enhanced. Thus, despite the remaining polyps seen in BAMLET treated Apc^{Min/+} mice at long-term follow up, there was no evidence of an ongoing response in those tumors, suggesting protection and a near healthy PD-1 phenotype. This observation and the lack of PD-1 signaling suggests that in addition to killing tumor cells, BAMLET treatment reprograms the local tumor tissue to a less aggressive state.

In addition, BAMLET treatment reduced Wnt/β-catenin signaling and β-catenin protein levels in the intestine. Remarkably, BAMLET treatment also affected major organs outside the intestine, reducing Wnt/β-catenin signaling and β-catenin staining in the lungs, livers and kidneys, potentially increasing the risk for oncogenic transformation. In parallel, major effects on these organs were detected, with fibrotic changes in the lungs, hyperlipidosis of the liver and changes to the renal cortex and papillae. In the lungs, these changes were accompanied by the formation of proliferating cell foci, projecting from the bronchial lining into the parenchyma. These foci stained for the lung adenocarcinoma marker TTF-1¹⁵, suggesting a lung origin rather than metastases from the intestinal tumors. The frequency and size of these tumor-like areas was reduced in BAMLET treated Apc^{Min/+} mice. The Wnt/β-catenin pathway is operative in adult lung epithelium and increased Wnt/β-catenin signaling may accompany epithelial cell injury and hyperplasia and impair epithelial-mesenchymal cross-talk in idiopathic pulmonary fibrosis (IPF)²⁶. Furthermore, patients with familial colon polyposis have been reported to develop lung cancer²⁷ suggesting that aberrant Wnt/β-catenin signaling may drive the development of tumors; a process that appears to be affected by BAMLET administration in the drinking water.

The effects of BAMLET on health parameters in tumor-free mice are notable. Positive effects on lipid metabolism, glucose metabolism and a reduction in insulin tolerance suggest a potential for BAMLET to accelerate lipid breakdown in intestinal tissues, reduce glucose levels and increase the insulin sensitivity of pancreatic tissues. The present study suggests that complexes formed by alpha-lactalbumin and oleic acid may provide fundamental health effects in the intestinal tract and in other organs, in addition to the beneficial therapeutic properties in cancer models. Taken together, these results suggest that peroral treatment may have far-reaching systemic effects, a potential paradigm shift for cancer prevention and therapeutic intervention.

### Methods

### Preparation of BAMLET and Alpha1-Oleate

The BAMLET complex was made by mixing bovine alpha-lactalbumin (Sigma, Cat #L5385) with oleic acid (Sigma, Cat #O1008). Alpha1 was synthesized using Fmoc solid phase chemistry (Mimotopes). The alpha1 sequence is: aa 1-39 Ac-KQFTKAELSQLLKDIDGYGGIA-LPELIATMFHTSGYDTQ-OH.

### Intestinal Cancer Model in Apc^{Min/+} Mice

Apc^{Min/+} mice were obtained from Jackson Laboratories at about eight weeks of age. Genotyping was performed by PCR analysis of genomic DNA obtained from blood collected from the retro-orbital sinus. Multiple tumors were developed in the small intestine at eight to ten weeks. Mice were acclimated for about two weeks at the local animal facility at BMC, Lund University in order to reduce stress from transportation.

For BAMLET therapeutic protocol, ten-week-old female mice (n=10 per group) were orally gavaged with 20 mg of BAMLET in 400 μl PBS, twice daily for ten days. Mice were not given water or food five hours prior to BAMLET administration. Food and water were provided 30 minutes after BAMLET oral administration. Sham treated mice were gavaged with 400 μl PBS. Mice were sacrificed two weeks and five weeks after the end of the treatment and intestinal tissue samples were collected for further analysis.

For BAMLET prophylaxis protocol, ten-week-old female Apc^{Min/+} mice (n=10 mice per group) were provided daily with BAMLET (20 mg/day in 5 ml PBS) in the drinking water for eight weeks and sacrificed at 18 weeks of age. A similar treatment was used in the survival study groups where mice were observed until 37 weeks of age.

### Tumor Enumeration and Sample Collection

Mice were sacrificed by isoflurane inhalation. Tumor enumeration and sample collection was presented as previously described²⁸. Tumor numbers and size were determined using a dissecting microscope (Olympus) and evaluated by three blinded investigators.

### Methylene Blue Staining

The opened intestinal segments were spread flat between sheets of filter paper and fixed overnight in 10% neutral buffered formalin. Formalin-fixed sections were transferred to 70% ethanol and stained with 0.2% methylene blue (Sigma, #M9140). Stained sections were rinsed in deionized water and imaged by a dissecting microscope.

### Histology and Immunohistochemistry

Swiss rolls of longitudinally opened intestinal segments were fixed overnight in 10% neutral buffered formalin. Samples were embedded in paraffin and 5 μm thick sections were further processed for histology and immunohistochemistry using antibodies for bovine alpha-Lactalbumin (ThermoFisher Scientific, Cat #A10-128A), β-catenin (CellSignaling, Cat #9562), Cyclin D1 (ThermoFisher Scientific, Cat #SC8396), Ki-67 (BD Biosciences, Cat #556003), VEGF (Abcam, Cat #ab46154), PD-1 (Abcam, Cat #ab214421) and TTF-1 (Abcam, Cat #ab227652) as previously described²⁸ with slight modifications. Citrate buffer (Dako Target Retrieval Solution, Agilent, Cat #S1699) was used for antigen retrieval for Cyclin D1, Ki-67, VEGF, bovine alpha-Lactalbumin and β-catenin staining. EDTA buffer (Abcam, Cat #ab64216) was used for antigen retrieval for TTF-1 staining. Immunohistochemistry was quantified by ImageJ. For Hematoxylin-Eosin (H&E) staining, Hematoxylin (ThermoFisher Scientific, Cat #7211) was used followed by Eosin-Y (ThermoFisher Scientific, Cat #7111) for counterstaining. Images were captured using the AX10 microscope or the Hamamatzu Nanozoomer scanner (Carl Zeiss) and the DAB-positive beta-catenin antibody fields (4-10) for each organ cross-sections were quantified by ImageJ.

For PD-1 immunofluorescence staining, paraffin sections were deparaffinized in xylene, rehydrated with reduced ethanol concentrations and then washed with deionized water. The slides were then immerged in target retrieval solution (Dako, S1699) and boiled for 20 minutes, followed by 30 minutes permeabilization with 0.25% Triton in PBS at room temperature. A blocking solution consisting in 5% goat serum in PBS was added on the sections for 1 h at room temperature, before adding the rabbit monoclonal anti-mouse PD-1 antibody (Abcam-ab214421, 1:150) in 1% goat serum and incubated overnight at 4° C. The slides were then washed with 0.025% PBS-T and stained with goat anti-rabbit Alexa Fluor-568 secondary antibody (1:200 for 1 h at room temperature, Invitrogen cat. n. A11034). The nuclei were counterstained with DAPI for 15 minutes, washed in PBS, and then mounted with Fluoromount aqueous mounting media (Sigma, F4680). Images were captured with the Hamamatzu Nanozoomer scanner and the fluorescence intensity was quantified by ImageJ.

### Real-Time In Vivo Fluorescence Imaging of BAMLET

BAMLET was labeled using VivoTag 680XL Protein Labeling Kit (Perkin Elmer). Apc^{Min/+} mice were orally gavaged with 10 mg of VivoTag 680-labelled BAMLET in 200 μl of PBS. The intestinal tissue samples were collected after 24 hours or 48 hours and imaged using an IVIS Spectrum imaging system (Perkin Elmer). BAMLET signal was acquired at fluorescent settings with 680 nm excitation.

### Transcriptomic Analysis

Approximately 5 mg of tissue was homogenized using a Tissuelyser (Qiagen) and total RNA was extracted using the RNeasy kit (Qiagen), amplified using a GeneChip 3'IVT Express Kit, hybridized onto Mouse Genome 430 PM array strips, and scanned using the GeneAtlas system (Affymetrix). Data was normalized using Robust Multi Average implemented in the Transcriptome Analysis Console software (v.4.0.1.36, Applied Biosystems, ThermoFisher Scientific). Relative expression was analyzed by ANOVA using the empirical Bayes method, and genes with an absolute fold change >1.5 or 2.0 were considered differentially expressed. Heat maps were constructed using Graphpad Prism 9 and differentially expressed genes were analyzed using Ingenuity Pathway Analysis software (IPA, Qiagen).

### Cell Culture

Colorectal adenocarcinoma cells (DLD1) and colorectal adenocarcinoma cells (HT29) were purchased from American Type Culture Collection (ATCC, VA, USA). A549 and DLD1 cells were cultured in RPMI-1640 supplemented with 1% non-essential amino acids, 1 mM sodium pyruvate, 50 μg/ml gentamicin and 5-10% fetal calf serum (FCS) at 37° C., 5% CO₂. All the cell culture reagents were purchased from ThermoFisher Scientific. Cells were sub-cultured every three days.

### Cell Death Assays

Two assays were used as an indirect measure of cell death. Luminescence-based ATPlite™ kit (Perkin Elmer) as well as the Prestoblue assay (ThermoFisher Scientific). Cells (5×10⁴ cells/well) were seeded in serum-free RPMI-1640 on 96-well plates and treated with BAMLET and alpha1-oleate at different concentration (7, 21, and 35 μM) and incubated for one hour. Afterward, FCS was added at concentration of 5% and the cells were continuously incubated for two hours at 37° C., at the end of which the two kits were used according to manufactures' instructions. Luminescence and fluorescence were measured using a microplate reader (Infinite F200, Tecan). The experiments were performed in triplicate and repeated twice.

### Colony Assay

Cells were seeded on 12-well plates (1×10³ cells/well) and incubated overnight. Cells were treated with different complexes: BAMLET (7, 21, and 35 μM) or alpha1-oleate (7, 21, and 35 μM) in serum-free media and incubated for one hour at 37° C., 5% CO₂. The incubation was continued after the addition of FCS to the media. On day ten post-treatment, the cells were washed once and fixed with cold methanol (300 μl) for 15 minutes on ice. Finally, colonies of cells were stained with hematoxylin (ThermoFisher Scientific, Cat #7211) for five minutes and images were captured under a dissecting microscope (Carl Zeiss). The experiment was repeated twice for each cell line.

### Live Cell Imaging Assay

To visualize the cellular uptake of BAMLET, cells were seeded on 6-well ibidi chambers (3.5×10⁴ cells/well) overnight and then treated with Janelia Fluor-549 labeled (TOCRIS, Cat #6147) BAMLET mixed with unlabelled BAMLET (21 μM) for one hour at 37° C. The nuclei were counterstained for five minutes with DAPI (Abcam Cat #ab228549, 1:1,000) and uptake of labelled BAMLET was captured with the LSM 900 laser scanning confocal microscope with oil immersion ×63 objectives (Carl Zeiss).

### Giant Unilamellar Vesicle Experiments

Giant unilamellar vesicles (GUVs) were formed by hydrogel-assisted swelling according to established protocols^{29,30}, with modifications previously described³¹. Briefly, glass cover slips were sonicated in 1 M NaOH solution (30 minutes), rinsed in Milli-Q water (three times) and further sonicated (30 minutes). Coverslips were plasma etched (1 min) using a BD-20 laboratory corona treater (Electro Technic Products Inc.) to render the surface clean and hydrophilic. A thin film of 1% (w/v) solution of molten ultra-low gelling temperature type IX-A agarose (Sigma) was deposited on the coverslip to provide a reaction bed for GUV formation. The cover slips were placed in AttoFluor® cell chambers (ThermoFisher Scientific). Following, 25 μl of egg phosphatidylcholine (#840051P—Avanti Polar Lipids) in chloroform (25 mg/ml) doped with 4% v/v rhodamine C (1 mg/ml, Sigma) were deposited onto the gelled agarose surface, and the solvent evaporated with nitrogen gas. The lipid-hydrogel film was rehydrated with 200 mM sucrose in PBS, pH 7.2 for one hour and then transferred into 200 mM glucose in PBS, pH 7.2 for sedimentation. GUVs were allowed to settle overnight before seeded on the coverslips for visualization after treatment with 21 μM BAMLET for one hour.

### TUNEL Assay

DNA fragmentation was detected using the terminal deoxynucleotidyl transferase dUTP nick end-labeling (TUNEL) assay (Click-IT TUNEL Alexa Fluor 488 imaging assay kit, ThermoFisher Scientific, #C10245). DLD1 cells were seeded in 8-well chamber slide (2×10⁴ cells/well) cultured overnight (37° C., 5% CO2) and incubated with BAMLET 7 μM, 21 μM and 35 μM for one hour in serum-free RPMI-1640 at 37° C. Cells were fixed (2% PFA, 15 minutes), permeabilized (0.25% Triton X-100 in PBS, 20 minutes) and incubated with TUNEL reaction mixture containing TdT for 60 minutes at 37° C. After the TUNEL reaction, cells were incubated with Click-iT reaction mixture for 30 minutes. Cells were counterstained with Hoechst 33342 (1:1000, 15 minutes, ThermoFisher Scientifi, Cat #62249), mounted in Fluoromount aqueous mounting media (Sigma, Cat #4680), and examined by LSM 900 confocal microscopy (Carl Zeiss). Fluorescence intensities were quantified by ImageJ.

### Statistical Analysis

All in vitro experiments were repeated at least two times. Data were expressed as means #S.E.M. The Gaussian distribution was determined by the D'Agostino-Pearson Test normality test. For data following a Gaussian distribution, student's t-tests were used. Other data sets were analyzed by Mann-Whitney U-test. Differences among control and treatment groups were determined by ANOVA followed by Tukey's multiple comparisons tests. Significance was assigned at * P<0.05, ** P<0.01 and *** P<0.001. Differences in survival were evaluated by Kaplan-Meier analysis with a Log-rank (Mantel-Cox) test.

### Study Approval

Experiments were approved by the Malmö/Lund Animal Experimental Ethics Committee at the Lund District Court, Sweden (#01302-20). Animal care and protocols followed institutional, national, and European Union guidelines and were governed by the European Parliament and Council Directive (2016/63, EU), the Swedish Animal Welfare Act (Djurskyddslagen 1988:534), the Swedish Welfare Ordinance (Djurskydssförordningen 1988:539) and Institutional Animal Care and Use Committee (IACUC) Guidelines.

### Example 1: Peroral BAMLET Treatment Reduces Intestinal Tumor Progression

Targeting locally growing tumors is essential to reduce the risk for tumor progression and metastatic disease. This study focused on tumor surveillance by the BAMLET complex in Apc^{Min/+} mice, which carry mutations relevant to hereditary and sporadic human colorectal cancer and develop intestinal polyps that progress to form large tumors.

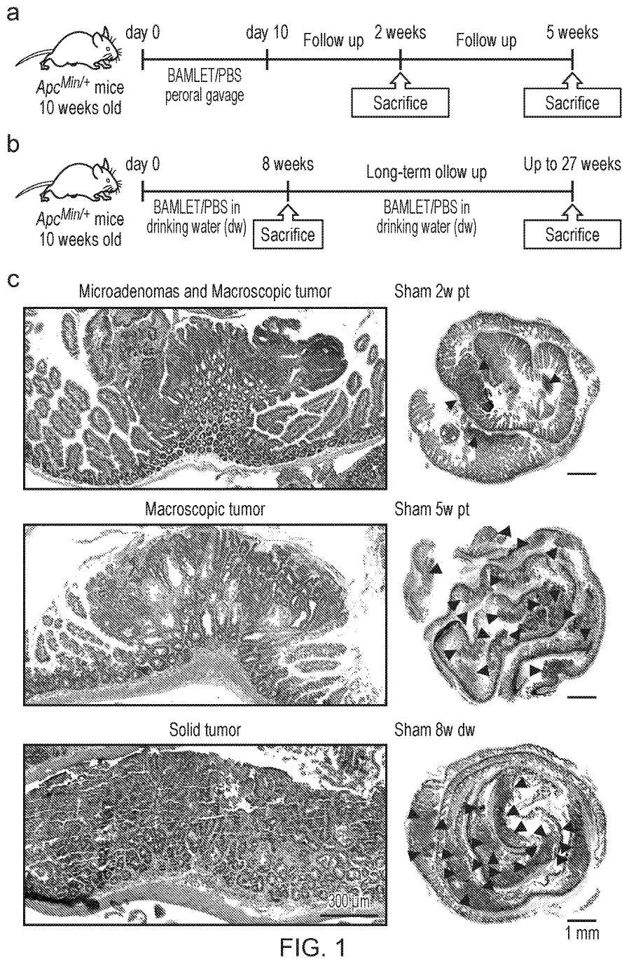

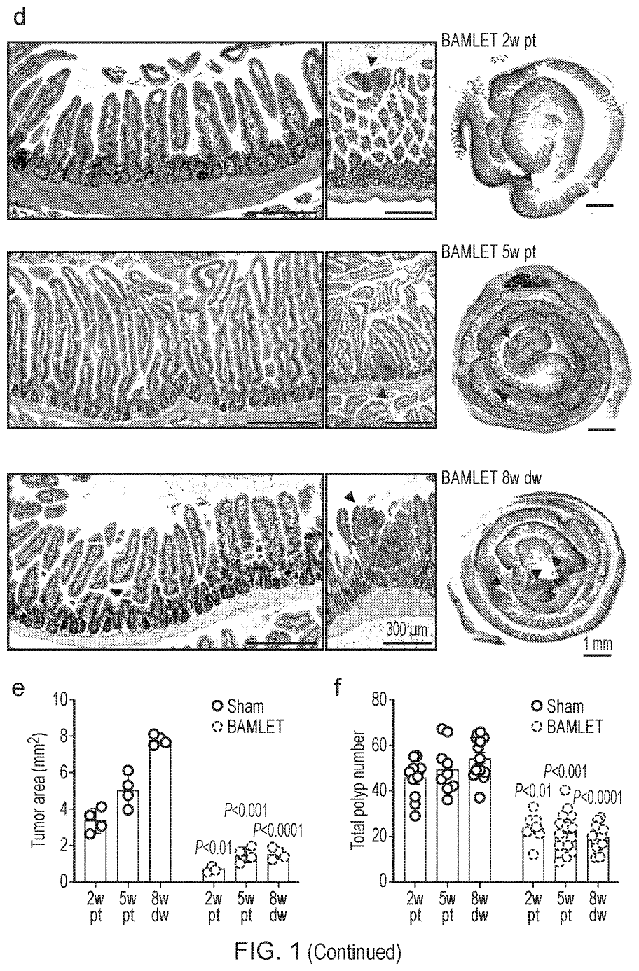

Two treatment protocols were used to administer BAMLET into the intestinal tract of Apc^{Min/+} mice, starting at ten weeks of age, when tumor formation has started (FIG. 1a, b). According to protocol 1, mice were subjected to peroral BAMLET gavage twice daily for ten days (FIG. 1a) and sacrificed two or five weeks post treatment. According to protocol 2, mice continuously received BAMLET in the drinking water and were sacrificed after eight weeks or followed long term until their health deteriorated. Control mice received PBS (sham group) (FIG. 1b).

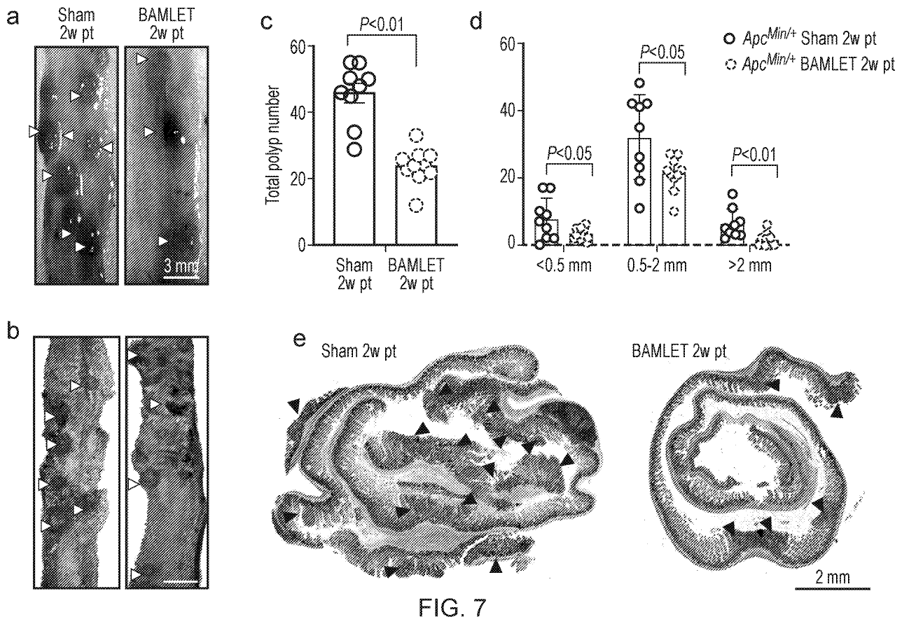

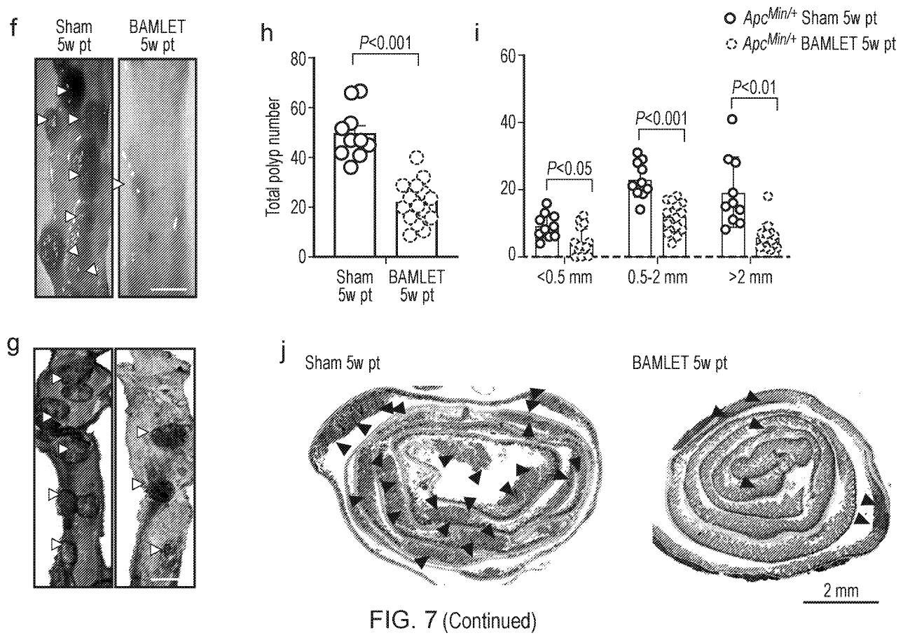

In this study all of the sham-treated Apc^{Min/+} mice developed intestinal tumors. Tumor progression was quantified as an increase in tumor number and size. Rapid tumor progression in sham treated mice was visualized by high-resolution imaging of H&E stained tissue sections, demonstrating an increase in tumor size from localized lesions (two weeks) and fully formed polyps (five weeks) to confluent tumors occupying most of the intestinal wall (FIG. 1c). The gradual increase in polyp number and area was further visualized in H&E stained “Swiss-roll” preparations of whole intestinal segments (FIG. 1c and FIG. 7). Polyps were counted by visual inspection and the counts were confirmed after methylene blue staining (FIG. 7).

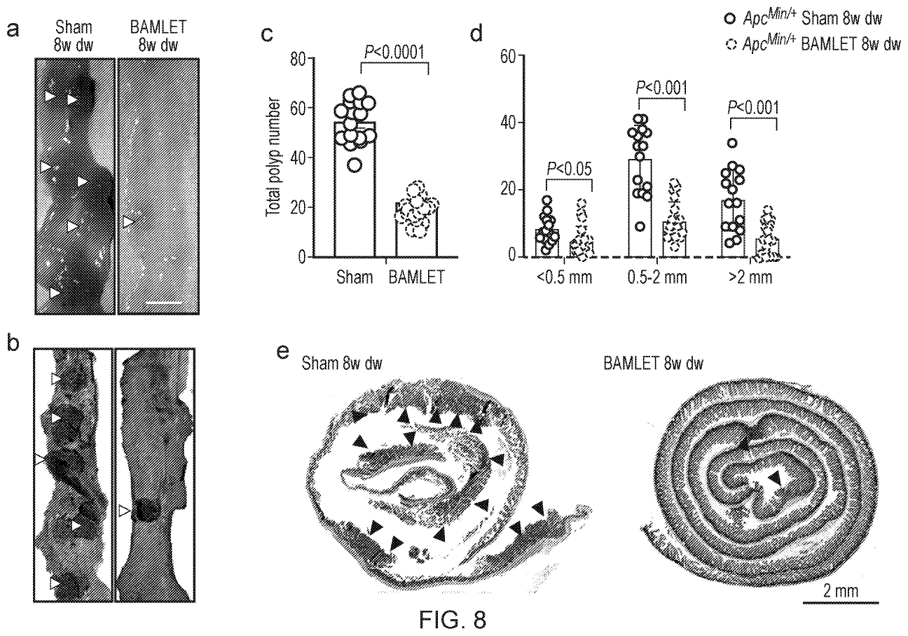

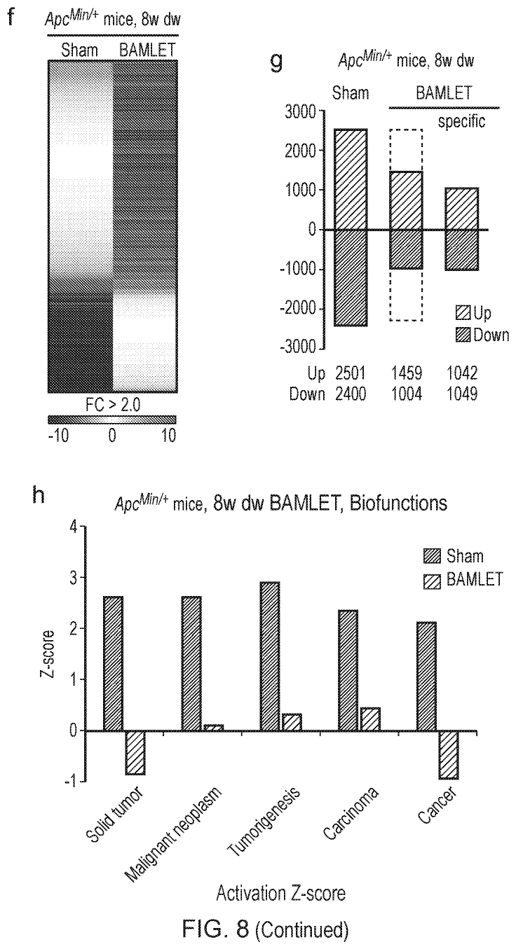

BAMLET treatment reduced the number of small tumors, fully formed polyps and confluent tumors along the intestinal wall (FIG. 1d). A normal villus structure was detected in the majority of sections after short-term BAMLET treatment (FIG. 1d). The total polyp number was lower after two and five weeks post oral gavage suggesting a rapid and lasting treatment effect (FIG. 1e, f and FIG. 7). BAMLET administration into the drinking water reproduced the protective effects of oral gavage (FIG. 1d). After eight weeks, the polyp number and the polyp size were markedly reduced, compared to the sham group (FIG. 1e, f and FIG. 8). The results suggest a potent anti-tumor effect of BAMLET, affecting established tumors and preventing tumor progression.

### Example 2: Inhibition by BAMLET of Cancer-Related Gene Expression

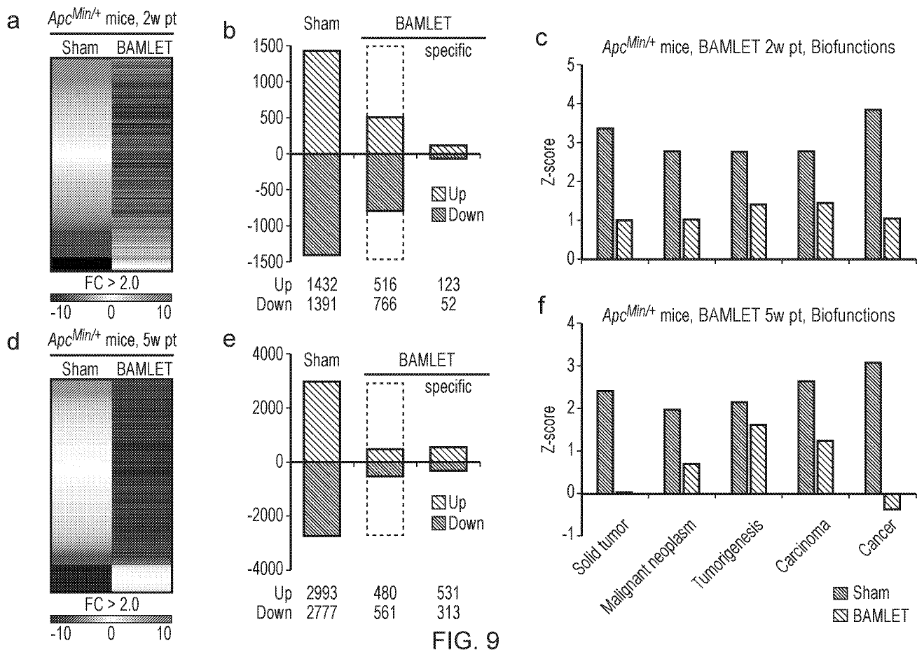

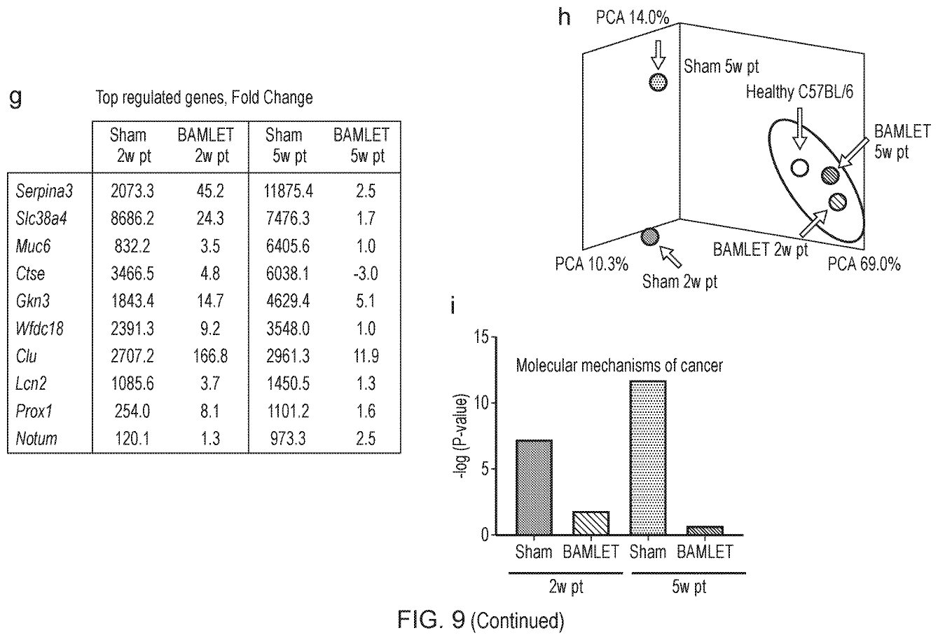

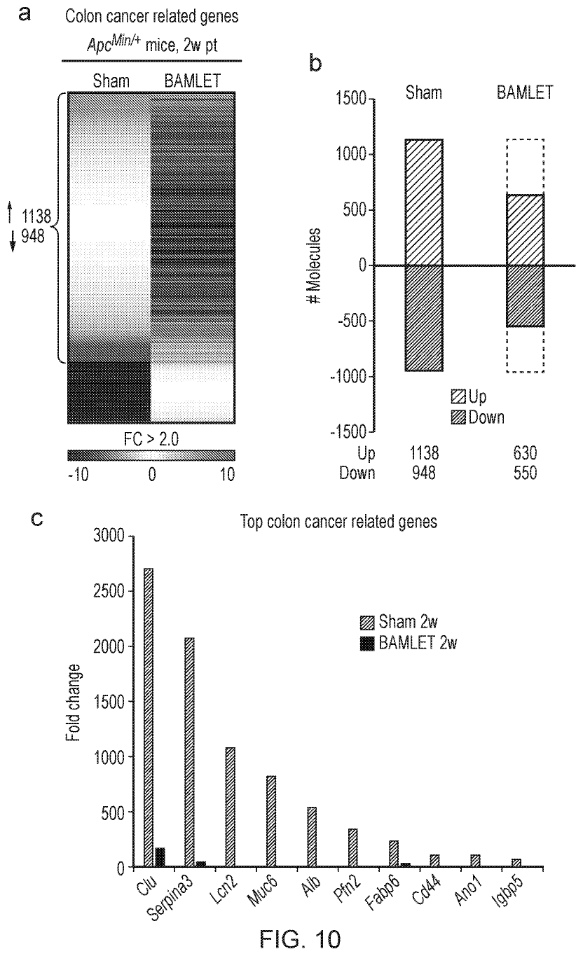

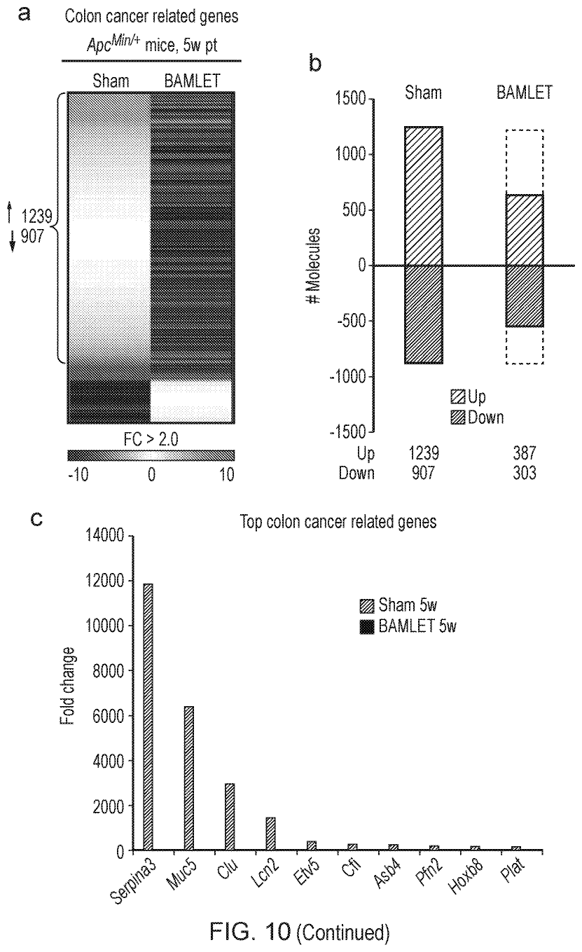

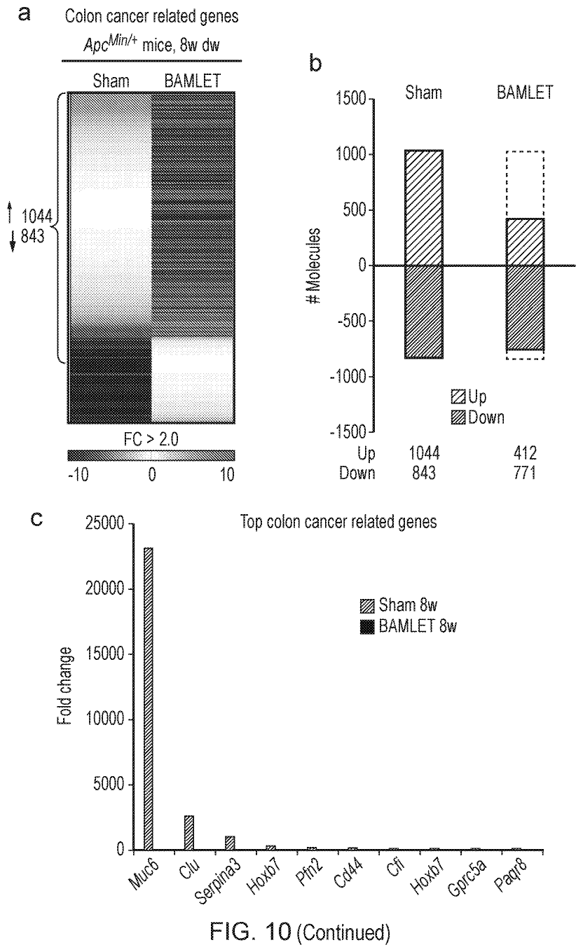

To characterize the tumor response to BAMLET, total intestinal RNA was subjected to whole-genome transcriptomic profiling. Gene expression profiles were compared, between the sham group with growing tumors and the BAMLET treated Apc^{Min/+} mice. Large numbers of genes were regulated in sham treated mice compared to healthy C57BL/6 mice, without intestinal tumors and top regulated biofunctions were all cancer-related (FIG. 8-10). In contrast, gene expression was significantly less affected in BAMLET treated Apc^{Min/+} mice including the cancer related biofunctions, which predominated in the sham group. By principal component analysis (PCA), BAMLET treated mice formed a cluster near the healthy mice and distant from the sham group, consistent with the lack of tumor development. The inhibitory effect included the molecular mechanisms of cancer gene network (FIGS. 8 and 9) and colon cancer related genes (FIG. 10).

### Example 3: Major Effects of BAMLET on the Tumor Environment

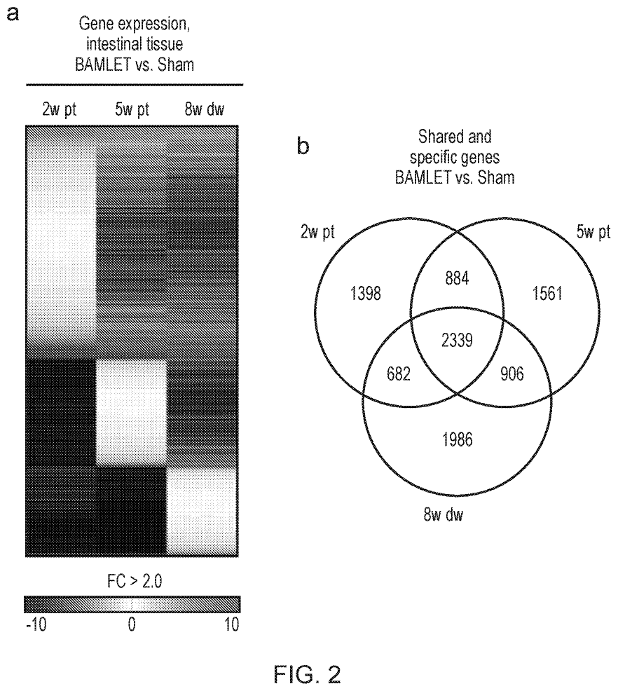

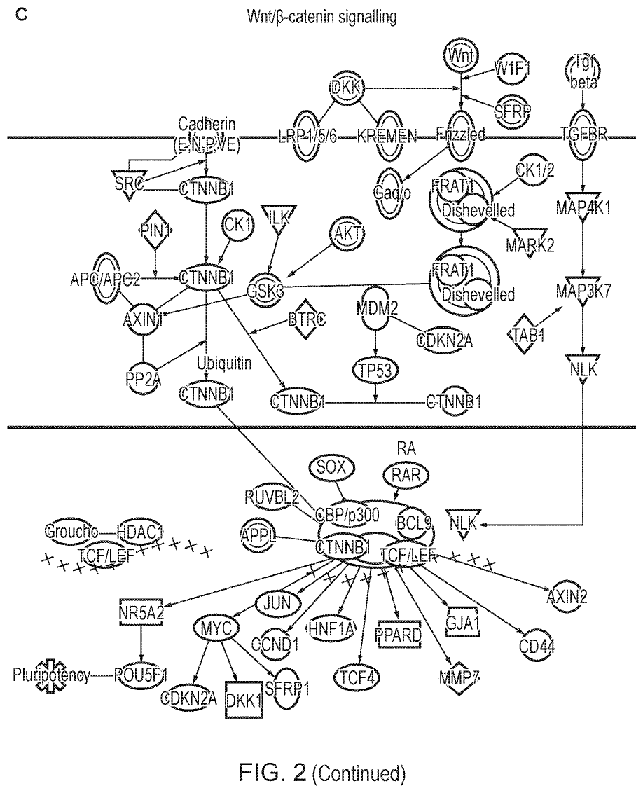

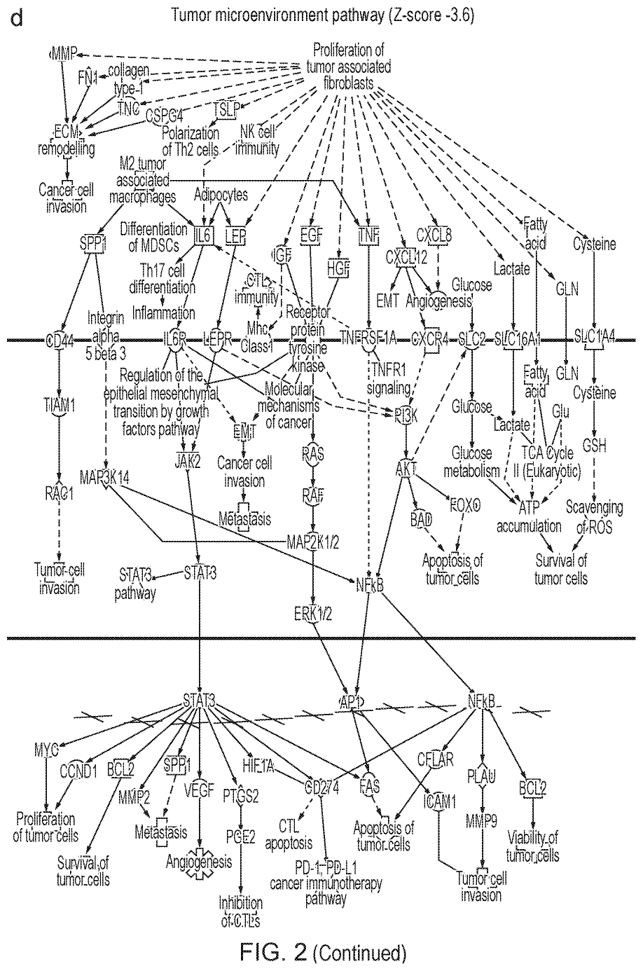

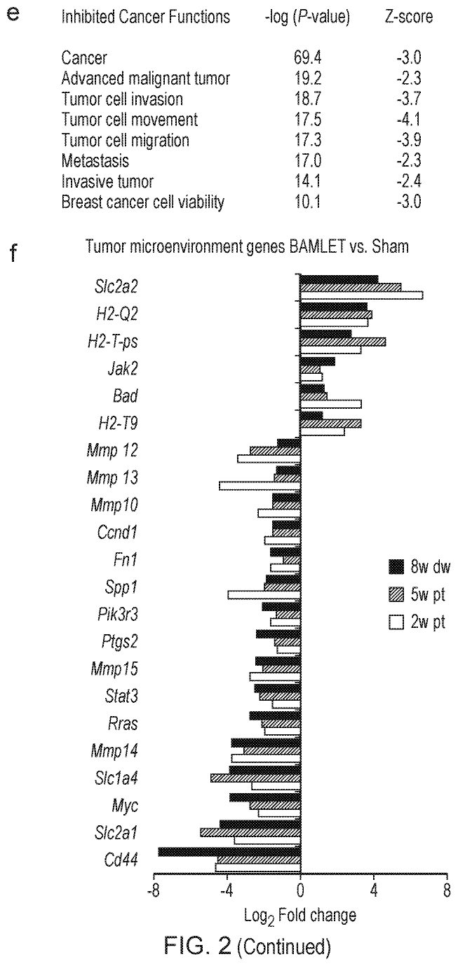

To further analyze the mechanism of protection, gene expression profiles were directly compared between BAMLET treated Apc^{Min/+} mice and the sham group (FIG. 2a, b). Strong treatment effects were documented as shared effects on gene expression in mice receiving BAMLET by oral gavage or in the drinking water. Regulated genes included the Wnt/β-catenin signaling pathway (FIG. 2c) as well as genes defining the tumor microenvironment, known to regulate angiogenesis, tumor cell mobility, tumor growth, metastasis and specifically colon cancer metastasis (FIG. 2d). Cancer-related biofunctions were inhibited (FIG. 2e) and the molecular mechanisms of cancer pathway genes were weakly expressed, including genes in the Ras signaling pathway. CD44 and its ligand, which binds SPP1 (osteopontin), were the most strongly inhibited genes in the tumor microenvironment pathway (FIG. 2f).

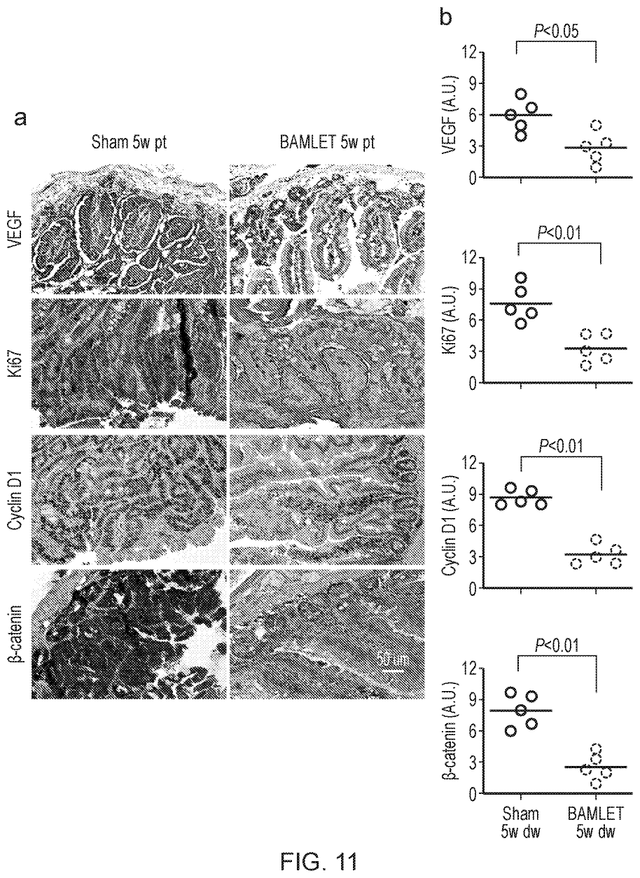

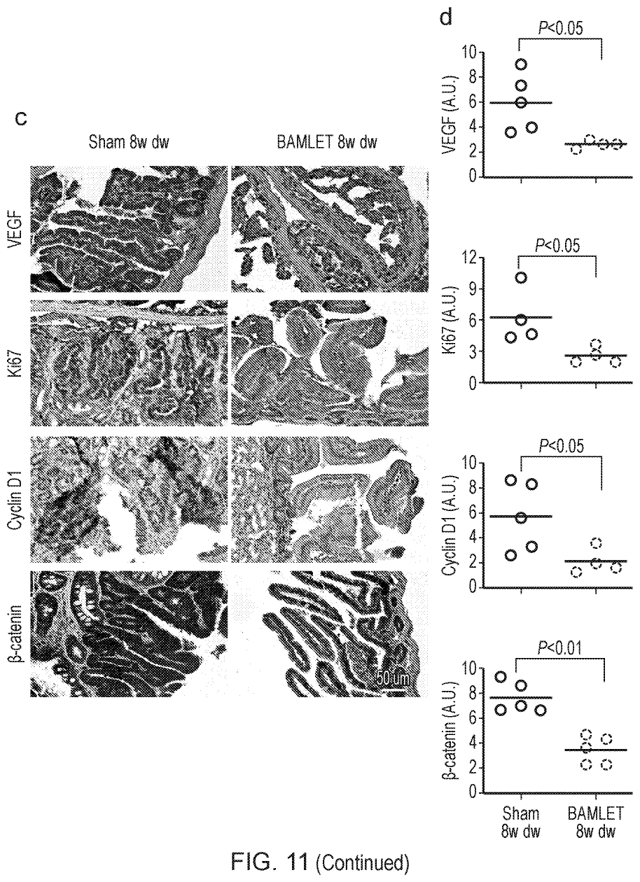

The protective effects were confirmed by staining of intestinal tissue sections for tumor markers vascular endothelial growth factor (VEGF), Ki67, Cyclin D1 and β-catenin (FIG. 11). All markers were less strongly expressed in the BAMLET treated Apc^{Min/+} mice groups, consistent with the lack of tumor development.

### Example 4: BAMLET is Internalized by Cancer Cells and Retained in Cancer Tissue

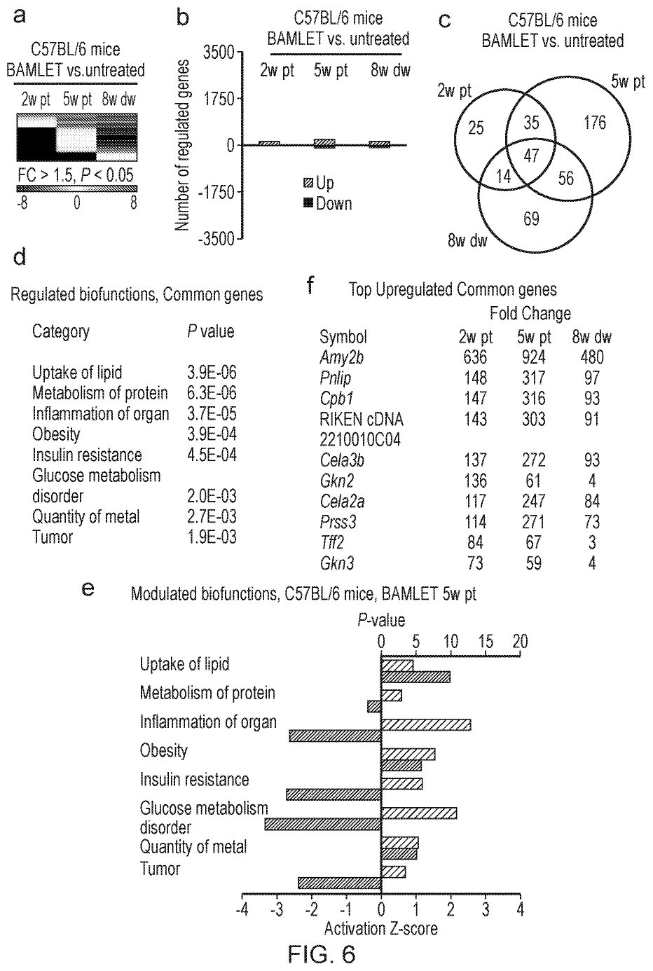

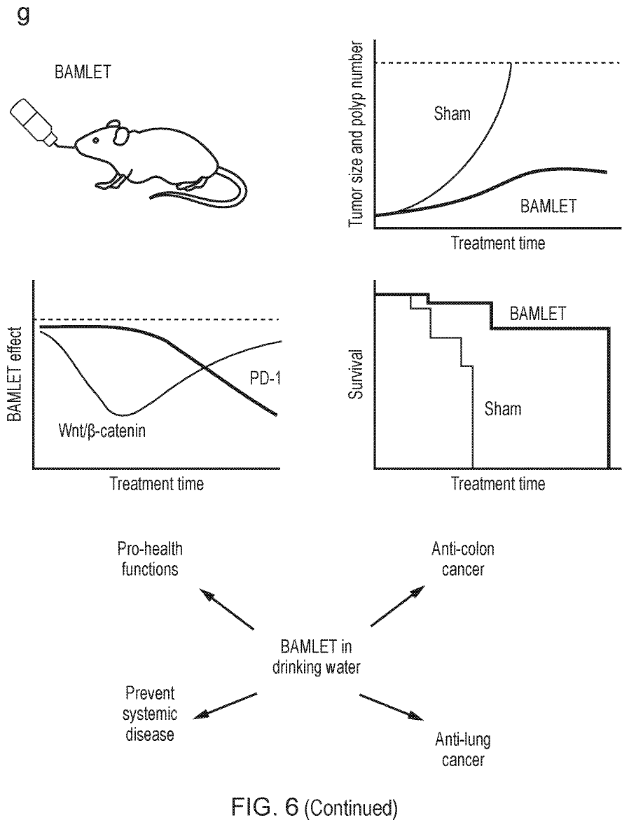

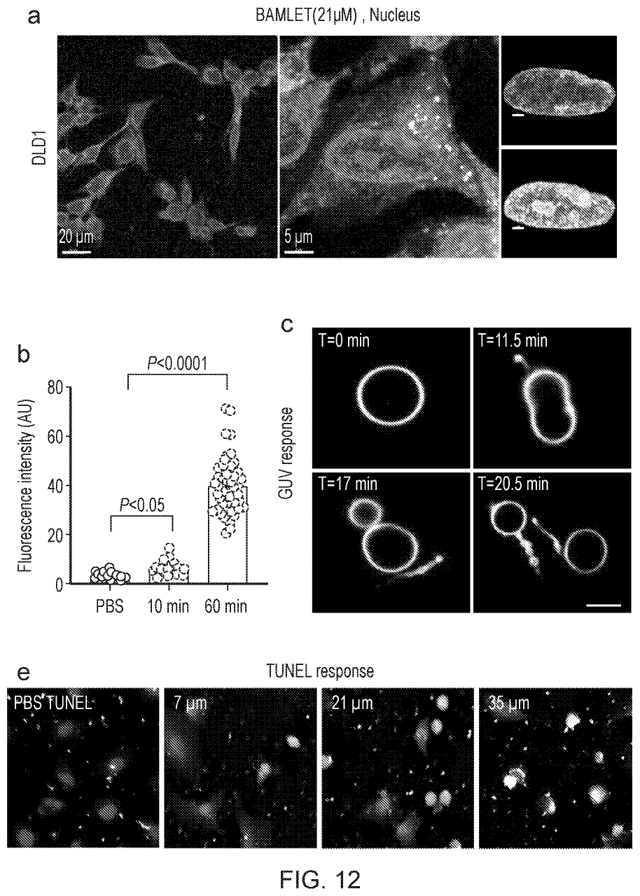

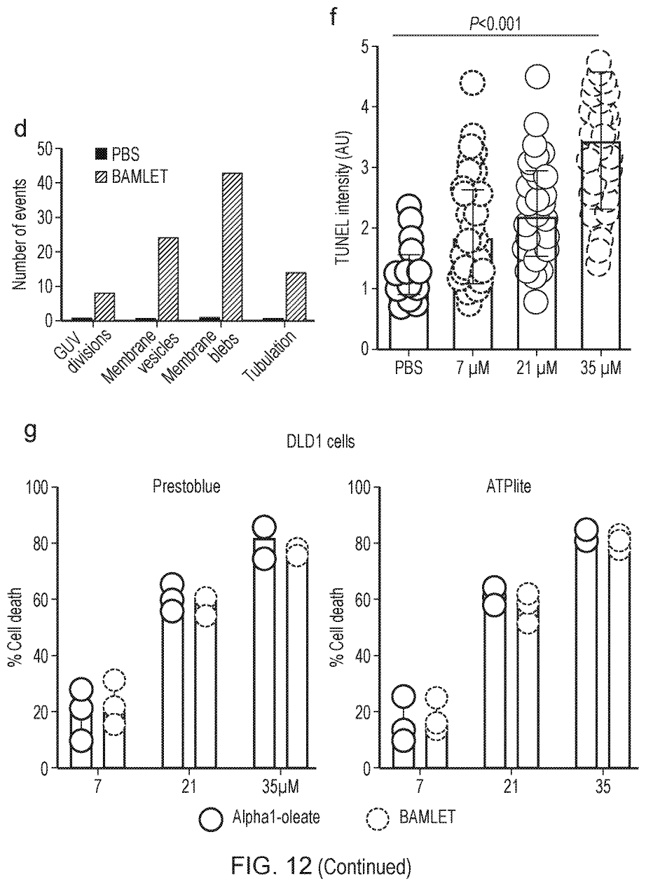

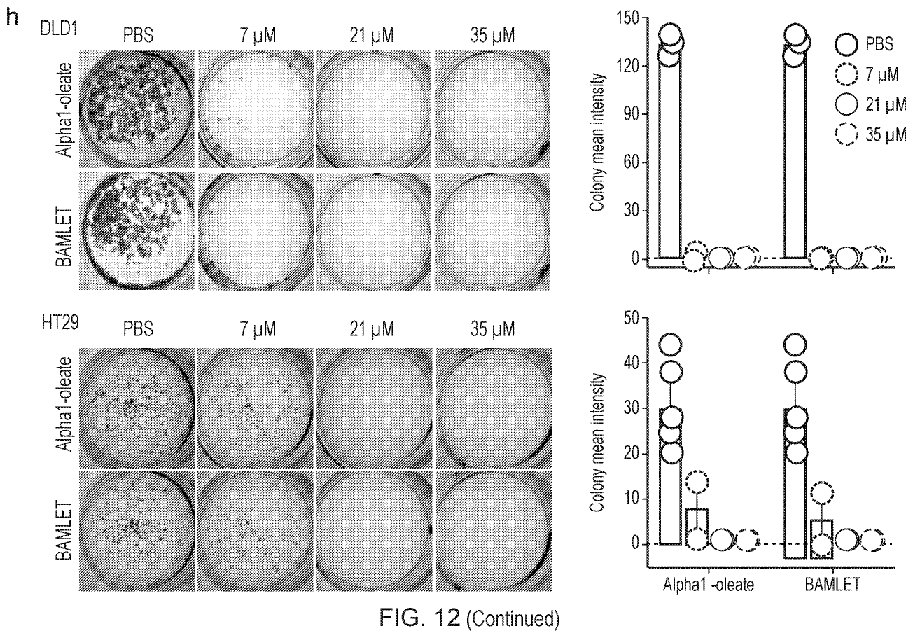

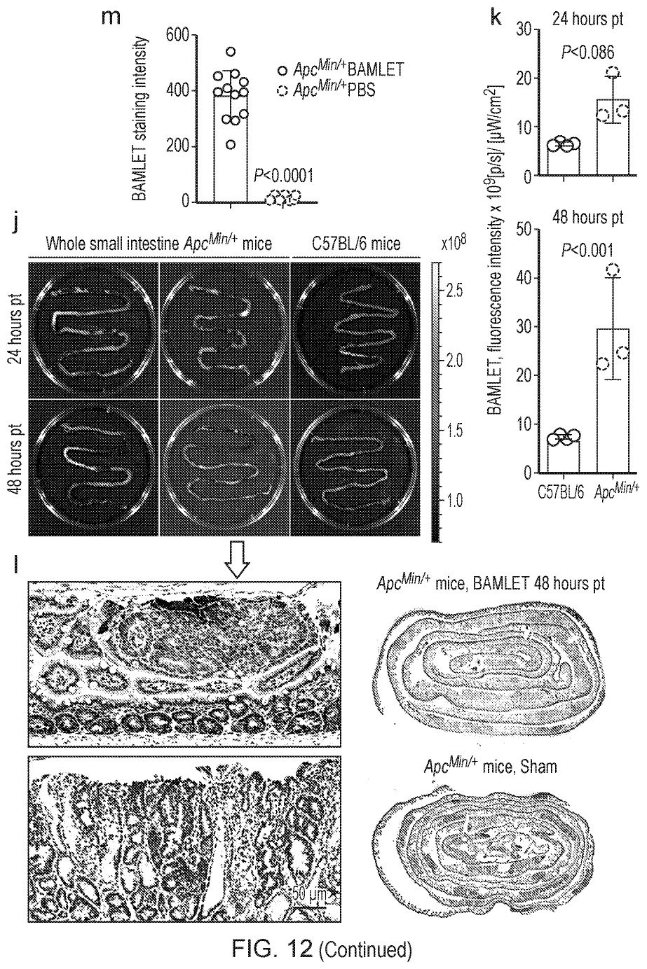

The efficacy of BAMLET treatment is consistent with a direct effect on emerging tumor cells as well as active reprogramming of the tissue environment. Cellular studies demonstrated direct, dose-dependent effects of BAMLET on colorectal adenocarcinoma cell lines (FIG. 12). BAMLET was rapidly internalized into the cytoplasm and nuclei of DLD1 cells (FIG. 12a, b) and a rapid membrane response to the complex was documented in giant unilamellar vesicles (GUV) composed of phosphatidylcholine, where BAMLET triggered rapid blebbing, tubulation and eventual vesicle division (FIG. 12c, d). An apoptosis like response was detected in treated cell nuclei, defined by DNA strand breaks (FIG. 12e, f). BAMLET triggered a rapid dose-dependent reduction in cell viability (FIG. 12g) and a lasting effect was documented in the colony assay, where growing, colony-forming cells were quantified after ten days (FIG. 12h, i). The tumoricidal effect of BAMLET was similar to that of the alpha1-oleate complex, which currently is used for clinical trials. Cell death was accompanied by DNA strand breaks detected by TUNEL staining, suggesting effects of BAMLET on the chromatin structure, also observed for HAMLET and alpha1-oleate (FIG. 6g, h). The cellular studies demonstrated a rapid and lasting, dose-dependent effect of BAMLET on colorectal adenocarcinoma cell viability.

To examine if BAMLET is retained in the intestine of tumor-bearing mice, VivoTag 680-labeled BAMLET was administered to 18-week-old APC^{Min/+} mice by oral gavage and monitored by whole body imaging. BAMLET treated healthy C57BL/6 mice were used as controls. Significant retention of BAMLET was detected in tumor bearing APC^{Min/+} mice after 24 and 48 hours but not in BAMLET treated C57BL/6 mice, suggesting that BAMLET is retained in intestinal tumors tissue in vivo (FIG. 12k, l). Intestinal tissue sections from BAMLET treated APC^{Min/+} mice were further subjected to immunohistochemistry, using BAMLET specific antibodies. BAMLET staining was detected in intestinal tissue sections from the BAMLET treated mice. Peripheral detachment of tumor fragments stained for BAMLET was detected in several sections (FIG. 12).

### Example 5: Long-Term Effects of BAMLET on Tumor Development and Systemic Disease

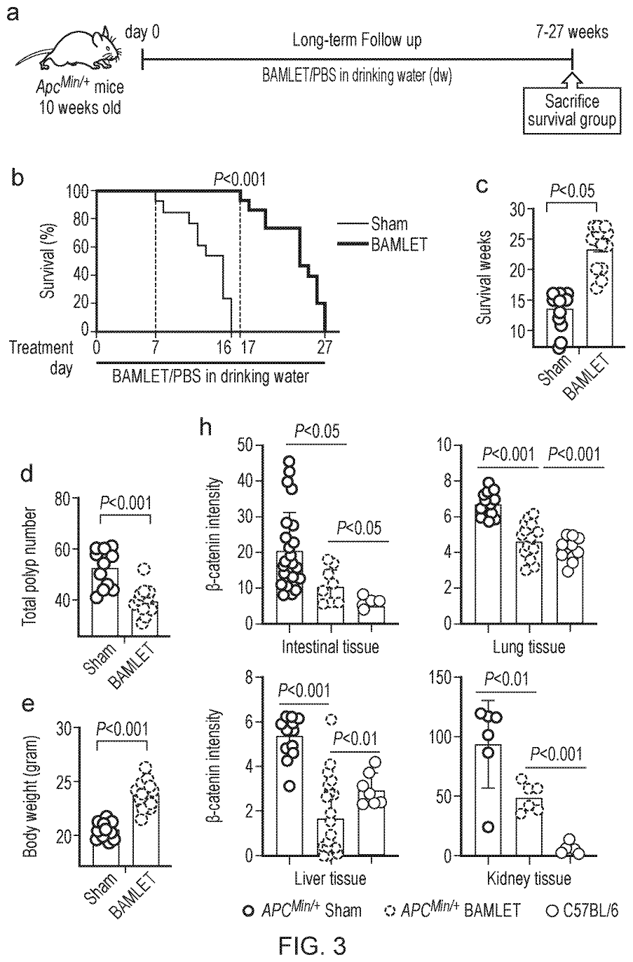

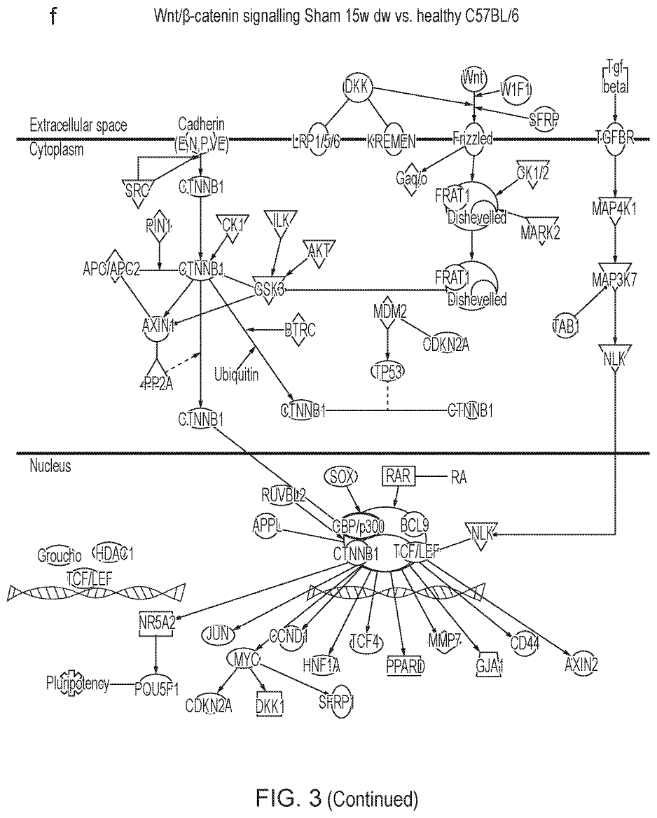

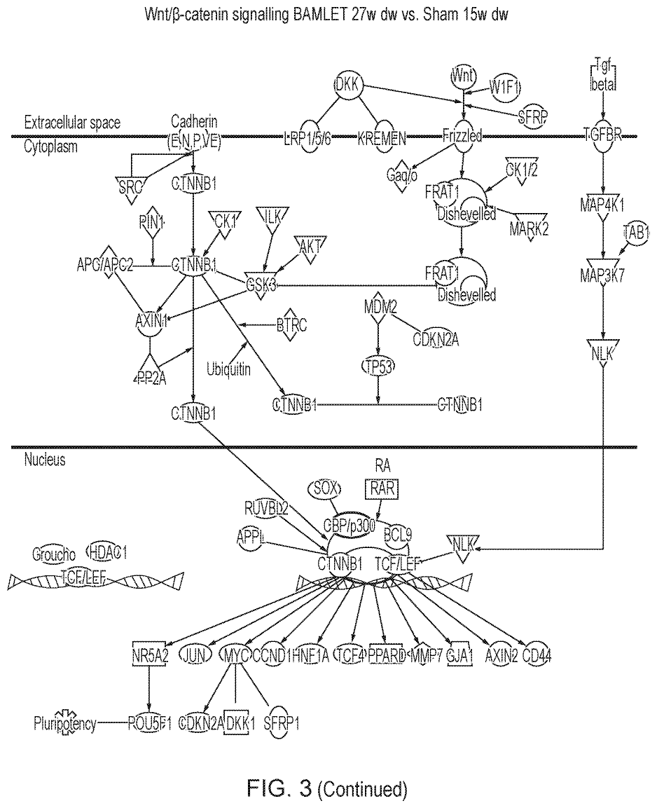

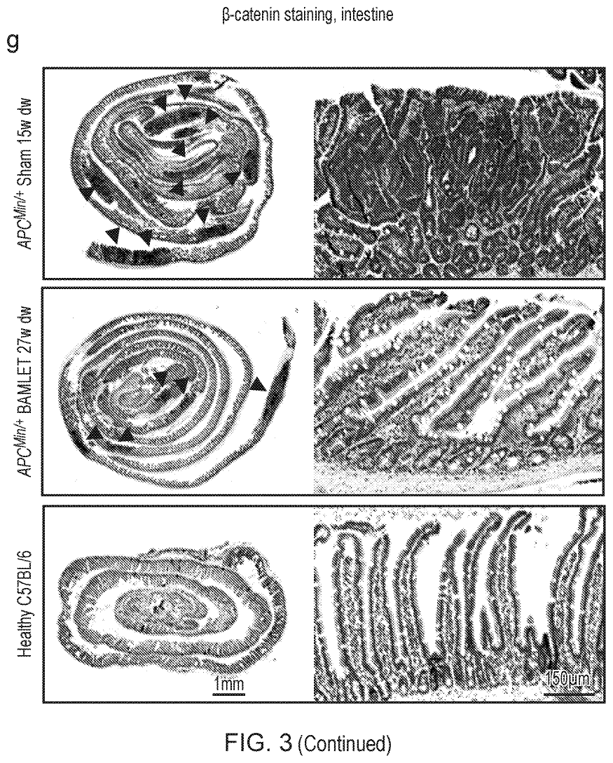

BAMLET supplementation of the drinking water had a lasting protective effect against tumor progression (FIG. 3a). By Kaplan-Meier analysis, an increase in survival was detected in BAMLET treated Apc^{Min/+} mice group compared to the sham group (FIG. 3b, c). Long-term BAMLET treatment reduced the total polyp number and polyp size and prevented the loss of body weight, compared to the sham group. (FIG. 3d, e).

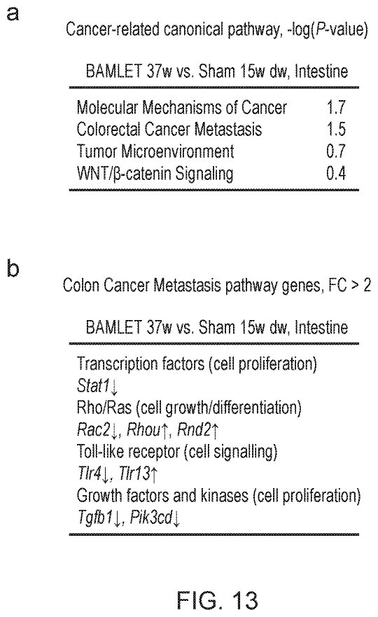

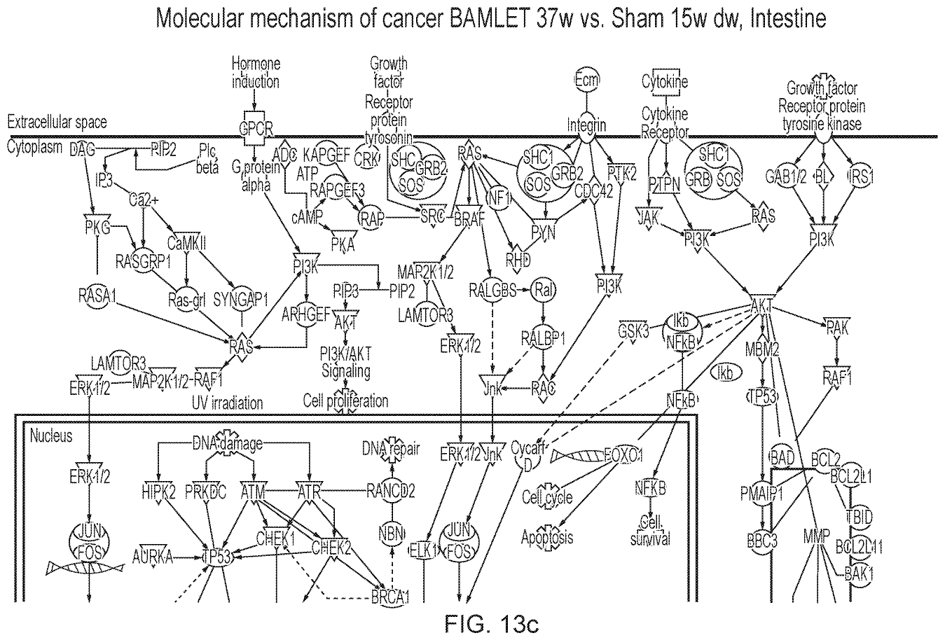

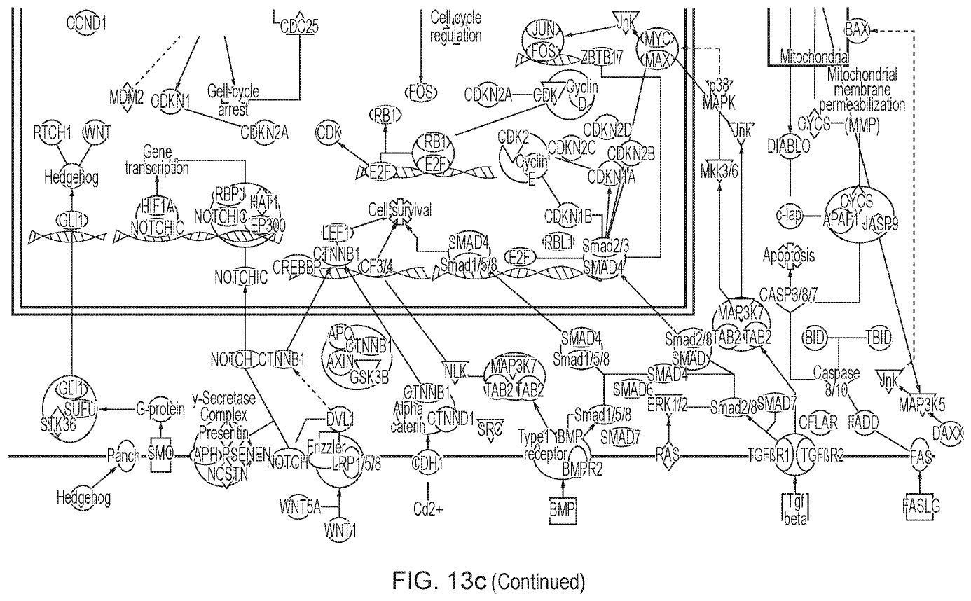

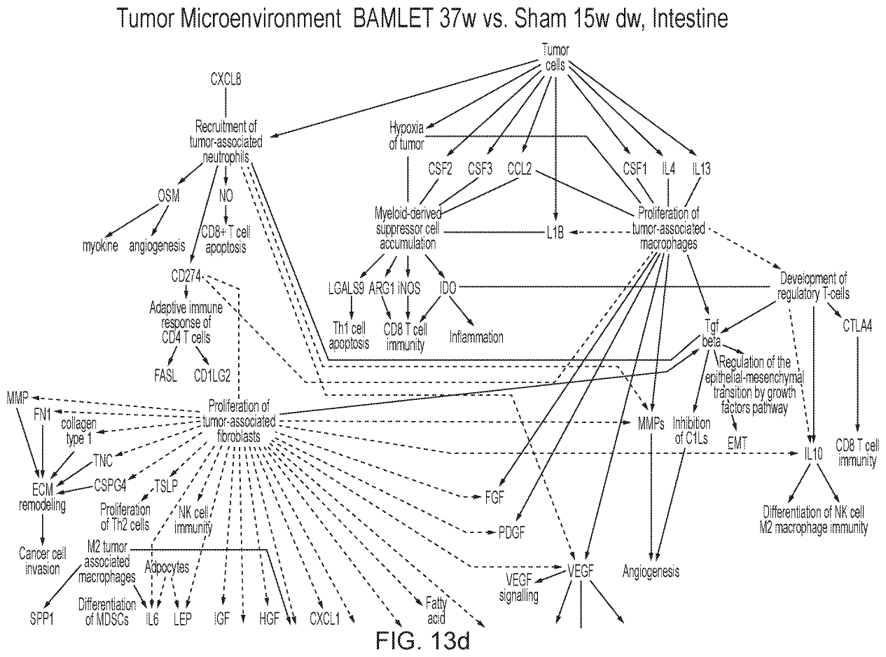

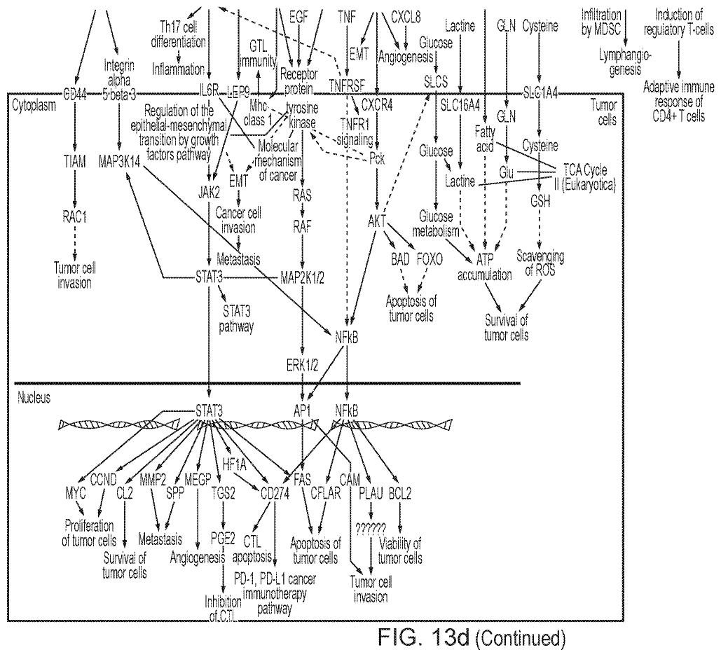

The intestinal gene expression profiles differed markedly between the sham treated and BAMLET treated Apc^{Min/+} mice groups at long-term follow up. This included the molecular mechanisms of cancer genes and the Wnt/β-catenin signaling pathway, which was upregulated in the sham group and inhibited in the BAMLET treated group (FIG. 3f, FIG. 13). Further support for this difference in gene expression was obtained by β-catenin staining, which was markedly reduced in the BAMLET treated mice (FIG. 3g), confirming the inhibition of the Wnt/β-catenin pathway by BAMLET at the protein level.

### Example 6: Inhibition of the PD-1 Signaling Pathway in BAMLET Treated Mice

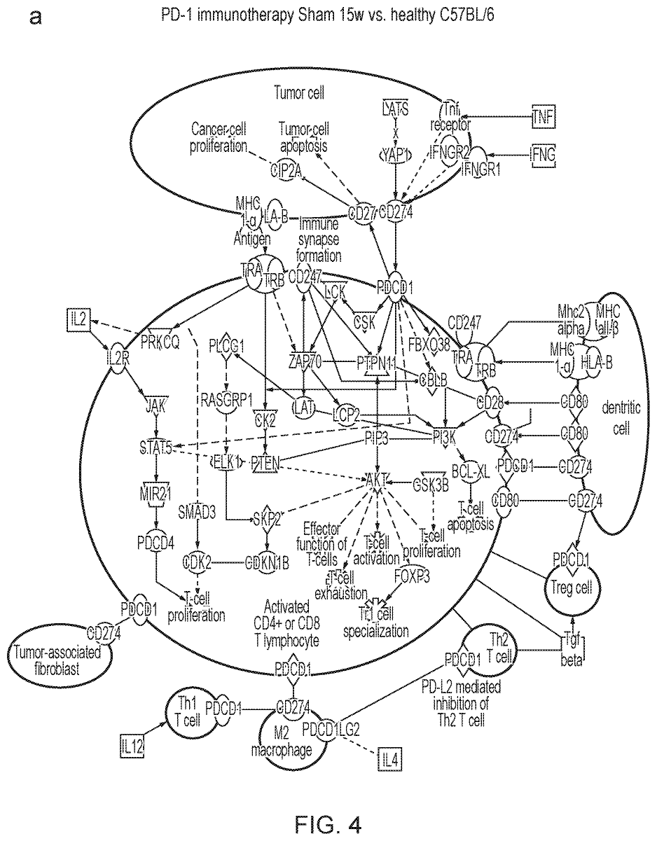

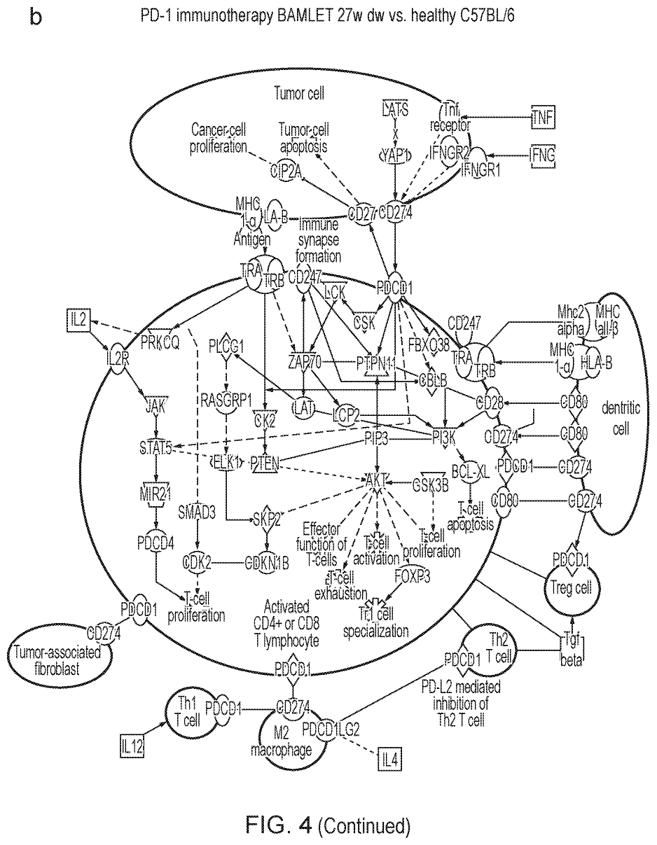

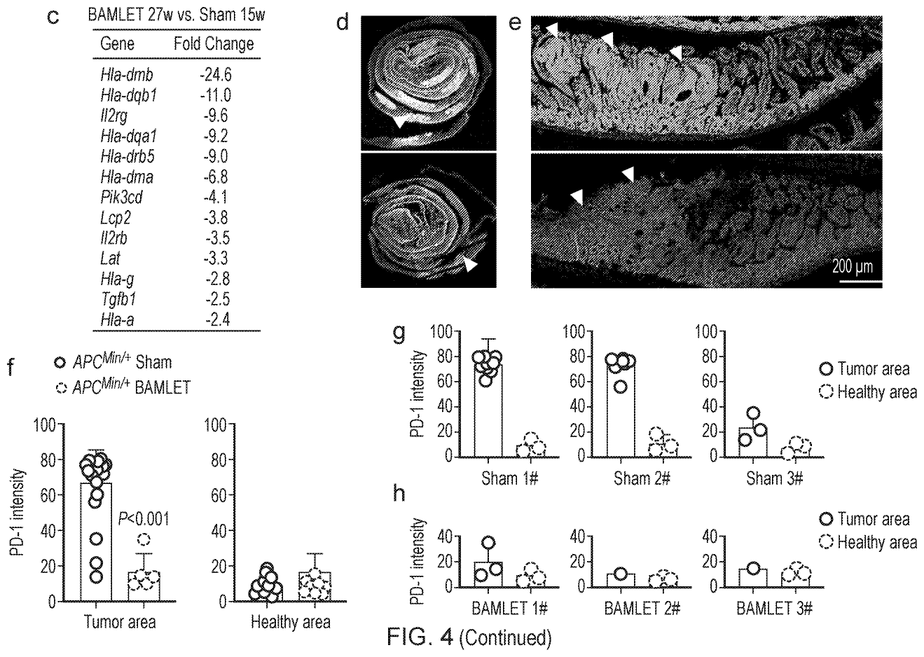

Gene expression analysis further identified the programmed death receptor 1 (PD-1) signaling pathway as upregulated in the sham group compared to healthy mice. Affected genes included the T cell related genes Rasgrp1, Cblb, Lcp2 (FIG. 4a). In contrast, BAMLET treated Apc^{Min/+} mice showed reduced PD-1 pathway activation in the tumor free and the tumor areas (FIG. 4b).

In a direct comparison between the BAMLET treated Apc^{Min/+} mice and the sham group, the PD-1 pathway was identified as the most strongly down-regulated (FIG. 4c). Genes related to the HLA class II histocompatibility antigens (Hla-dmb, Hla-dqb1, Hla-dqa1, Hla-drb5 and Hla-dma), IL2 receptors (IL2rg, Il2rb) and growth factor (Tgfb1) were down-regulated in BAMLET treated Apc^{Min/+} mice compared to sham (FIG. 4c).

PD-1 staining was clearly detected by immunohistochemistry in the sham group and was more pronounced in tumor areas than in adjacent healthy tissues (FIG. 4d). In contrast, PD-1 staining was significantly lower in the BAMLET treated Apc^{Min/+} mice, in tumor and healthy tissue areas, suggesting an effect of BAMLET on PD-1 at the protein level.

### Example 7: Evidence of Protection Against Systemic Disease

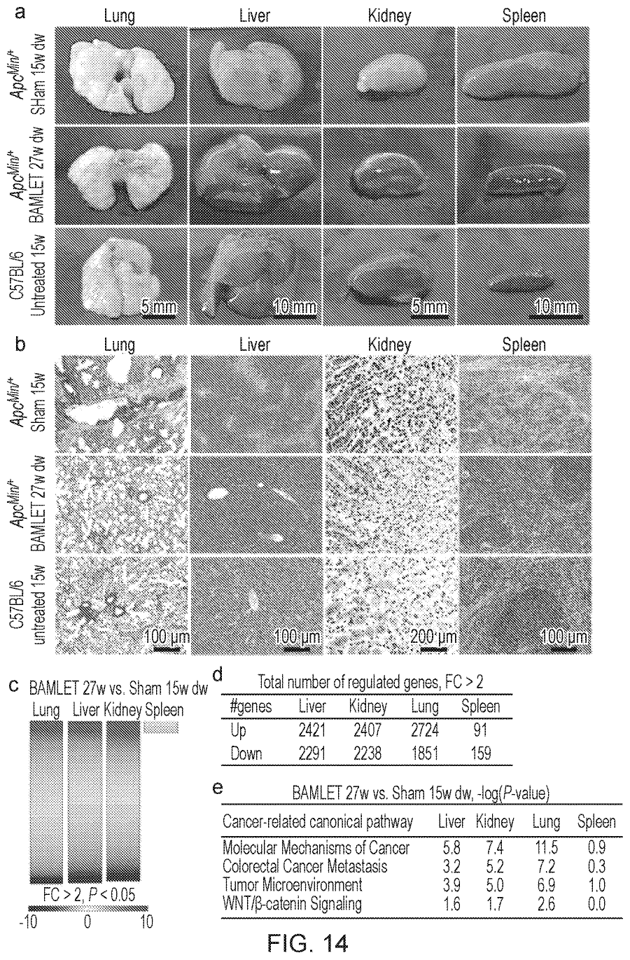

At follow up, macroscopic changes were observed in the intestine, the peritoneal cavity and major organs. The intestines and spleens were enlarged, the livers and kidneys were discolored, and the lungs were pale and appeared solid. These changes were much less apparent in the BAMLET treated Apc^{Min/+} mice except for moderate enlargement of the spleens in individual mice (FIG. 14a).

The disease response was further examined by histopathology. Lungs from sham treated Apc^{Min/+} mice showed evidence of thickened alveolar septa and reduced alveolar spaces, suggesting hypercellularity or focal collapse of lung parenchyma. The liver tissue showed evidence of centrilobular micro- and macro-vacuolar steatosis or “fatty liver” and binucleated hepatocytes were observed. Spleens in the sham group showed a loss of lymphoid foci and a more chaotic arrangement of lymphoid cells (FIG. 14b).

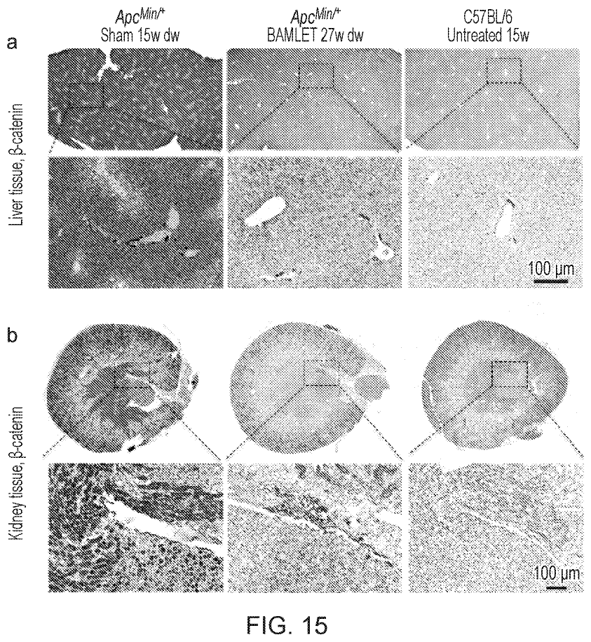

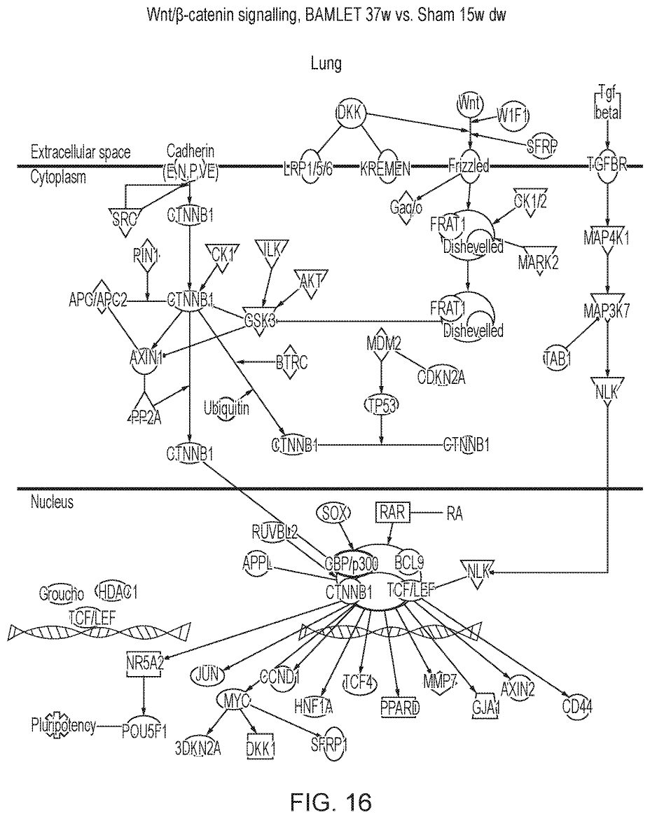

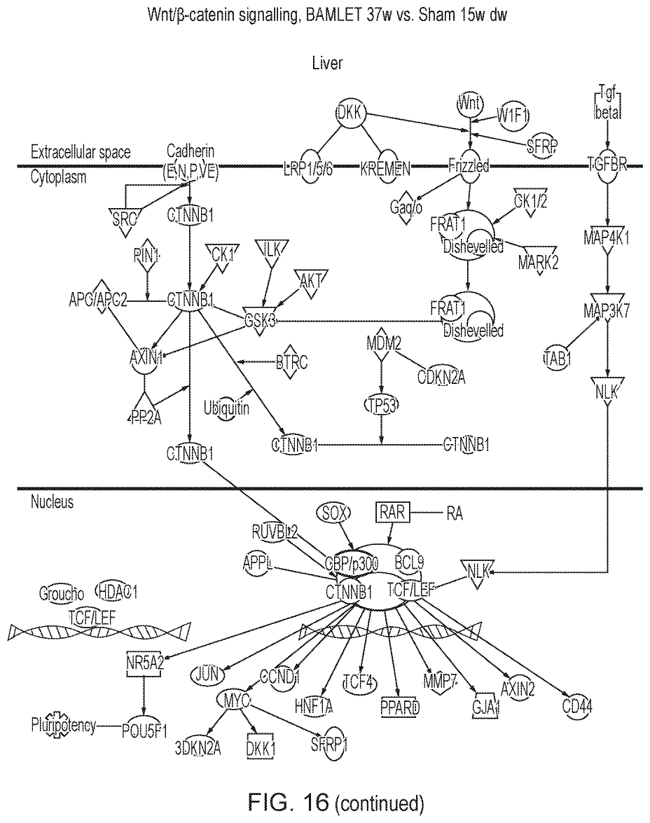

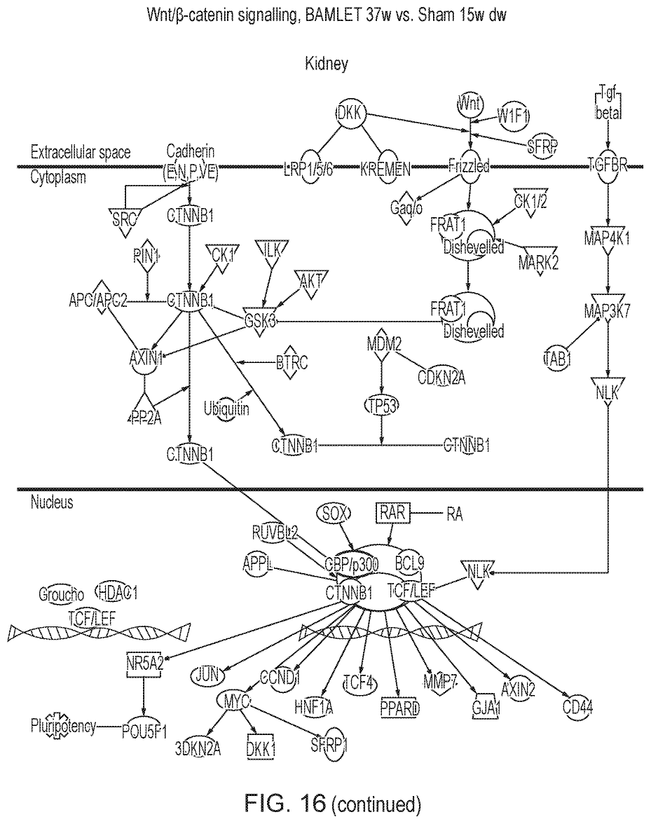

The systemic disease response was accompanied by an increase in β-catenin staining in the different organs (FIG. 15). In sham treated Apc^{Min/+} mice, staining was intense in the multi-layered lining of the bronchial tree and in the thickened septa between the alveoli. Focal cell aggregates were also formed along the renal pelvis, as well as a higher overall staining intensity in the renal papillae. Intense, diffuse β-catenin staining was further detected in the livers of the sham treated Apc^{Min/+} mice. In contrast, BAMLET treated Apc^{Min/+} mice showed a general reduction in β-catenin staining in all tissues, suggesting treatment effects outside of the intestinal compartment. BAMLET staining was not detected in lungs, livers or kidneys, in contrast to the intestine (FIG. 15).

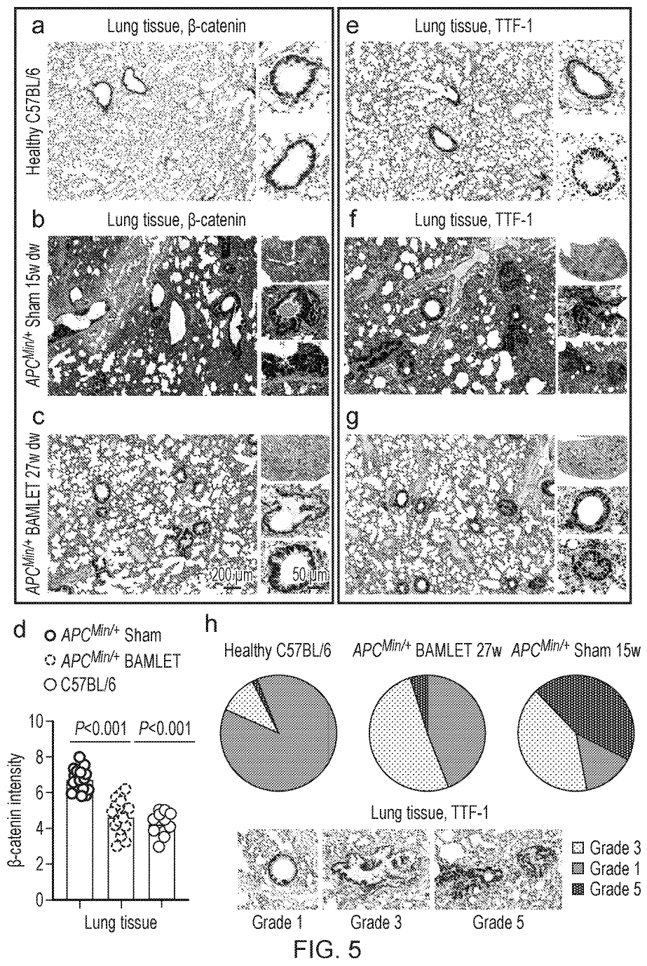

β-catenin staining further detected highly stained areas in the lungs of sham treated APC^{Min/+} mice, corresponding to cross-sections of the bronchi. A pattern of cell proliferation was detected in these areas creating a multilayered bronchial wall and areas of cell cluster apparently spreading from the bronchial wall, suggesting tumor formation (FIG. 5a-d). Further staining was performed using antibodies to Thyroid transcription factor (TTF-1), which is highly expressed in lung adenocarcinoma and used as a diagnostic marker for lung cancer¹⁵. TTF-1 was strongly expressed by the proliferating cells and TTF-1 staining overlapped with β-catenin staining (FIG. 5). The number of areas with proliferating cells was markedly reduced in the BAMLET treated Apc^{Min/+} mice, as well as the level of TTF-1 staining in those areas (FIG. 5e).

The systemic treatment effects were confirmed by gene expression analysis comparing the sham group to BAMLET treated Apc^{Min/+} mice (FIG. 14, 16). The molecular mechanism of cancer network was upregulated in lungs, liver and kidneys of sham treated mice compared to the BAMLET treated Apc^{Min/+} mice group and differences were further observed for the colon cancer metastasis genes and tumor microenvironment pathways in these organs (FIG. 14).

The results suggest that in addition to the anti-tumor effects in the intestine, BAMLET treatment may condition other tissues to become less prone to cancer development by the inhibition of the molecular mechanisms of cancer pathways and of pro-metastatic genes in the tumor microenvironment network.

### Example 8: Lack of Toxicity in Healthy Mice and Beneficial Health Effects

To identify effects on healthy tissues, healthy C57BL/6 mice received BAMLET by oral gavage or in the drinking water (FIG. 6). The effects were evaluated by macroscopic inspection of intestinal tissues and gene expression analysis. There was no evidence of inflammatory or necrotic changes in the intestines or peripheral organs in these mice, compared to untreated controls. The number of regulated genes was low (about 150 genes) with no evidence of a toxic response (FIG. 6a-c). Interestingly, genes involved in lipid metabolism, glucose metabolism, insulin tolerance and immune regulation were significantly affected in the BAMLET group (FIG. 6d, e). Top up-regulated genes included genes encoding amylases that are important for carbohydrate digestion (Amy2b), lipases for lipid digestion (Pnlip) and several proteases for protein digestion (Cpb1, Prss3, Cela3b, Cele2a) (FIG. 6f). No changes in macroscopic appearance were detected in tissues outside the intestinal tract and there was no change in organ weights in healthy mice exposed to BAMLET.

### DISCUSSION

The inventors examined the potential of BAMLET as a peroral tumor surveillance molecule, by evaluating its preventive and therapeutic effects on intestinal tumor development and extra-intestinal organs in tumor-prone Apc^{Min/+} mice. While strong anti-tumor effects were demonstrated in these mice, healthy C57BL/6 mice were virtually unresponsive to BAMLET, except for effects on lipid and glucose metabolism. The findings illustrate how a single protein complex may solve multiple, essential needs of the host, in this case the synthesis of lactose in the mammary gland, the purging of cancer cells from the intestinal tract and extra-intestinal tissues and the metabolic effects in healthy mice. Without being bound by theory, the inventors consider that this mechanism may have evolved to rid the intestinal mucosa of the newborn of virus-transformed cells and immature cells that resemble cancer cells. The findings further emphasize the potential of BAMLET as a new prophylactic or therapeutic tool in cancer treatment.

The molecular basis of these therapeutic effects was investigated by gene expression analysis. Genes that define the tumor microenvironment were strongly upregulated in this model of spontaneous cancer formation but not expressed or weakly expressed in the BAMLET-treated group at several early time points. A lasting effect of BAMLET treatment five weeks post oral gavage and genes known to drive metastasis, tumor growth and angiogenesis were strongly downregulated. In addition, the Wnt/β-catenin signaling pathway was inhibited, suggesting that BAMLET may protect the tissue environment by preventing major, cancer-related gene networks from being expressed. While these effects may reflect the delay in tumor development observed in the BAMLET-treated mice, the results further suggest that BAMLET actively promotes cellular differentiation and restores tissue homeostasis by instructing aberrantly proliferating cells to return to normality. Furthermore, some of these effects were still detected after long-term follow-up, suggesting that BAMLET administration in the drinking water maintains an antitumor and pro-differentiation pressure by removing emergent cancer cells and reprogramming gene expression in intestinal tissues.

Notably, BAMLET treatment also affected major organs outside the intestine, reducing and β-catenin levels in the lungs, liver and kidneys, strongly affecting genes defining the tumor environment and cancer related genes, potentially reducing the risk for oncogenic transformation and tumor development. The extra-intestinal effects of BAMLET treatment were unexpected, as the Apc^{Min/+} model normally is used as a model of colon cancer metastasis but most mice die of anemia or intussusception before progression with an expected life span of about 100 days²⁴. In BAMLET-treated mice, survival was significantly extended compared to the sham-treated group which survived up to 180 days. TTF-1 staining of the lungs²³ suggesting a lung origin of the proliferating cell foci rather than metastases from the intestinal tumors. The Wnt/β-catenin pathway is operative in the adult lung epithelium²⁵ and, patients with familial adenomatous polyposis have been reported to develop lung cancer²⁶, suggesting that aberrant Wnt/β-catenin signaling may drive the development of tumors, a process that appeared to be affected by BAMLET administration in drinking water. The tumoricidal effect of BAMLET and the related complexes HAMLET and alpha1-oleate are not limited to specific cancer types. Treatment effects on bladder cancer and skin papillomas have been demonstrated in controlled trials and in animal models.

Unexpectedly, the PD-1 signaling pathway was inhibited by BAMLET treatment and PD-1 protein levels were markedly reduced in the treated mice compared to the sham group. The findings suggest that in addition to directly affecting the tumors, BAMLET may inhibit the PD-1 feedback loop and the inhibition of T cell responses against tumors. Except for the PD-1 associated human leukocyte antigens (HLA) antigens, which were inhibited, there was no evidence of a more general effect of BAMLET on the immune response of intestinal tissue. The functional consequences of this observation are not clear, as the efficacy of immune checkpoint inhibitors, such as anti-PD-1/PD-L1 therapy, is limited for the treatment of colorectal cancer and restricted to the treatment of microsatellite instability-high (MSI-H) tumors. For patients with microsatellite stability (MSS) colorectal cancer, approximately 90% of patients, the response rate is only 5%-10%.

Healthy C57BL/6 mice were remarkably unresponsive to BAMLET treatment, emphasized the affinity of the complex for tumor tissue rather than healthy tissue. There was no evidence of toxicity, defined by macroscopic inspection or behavioral change and a lack of BAMLET retention in the intestines of healthy mice. Positive effects suggested that BAMLET may be support physiologic processes in normal tissues. These included effects on lipid metabolism, glucose metabolism and insulin tolerance. The results highlight the functional diversity of alpha-lactalbumin. It may be speculated that evolution favors proteins that adapt their structure to solve multiple functional challenges. The oleic acid-bound protein is partially unfolded, exposing different functional domains than the native protein and achieving a different but equally essential function to support tissue development. The structural and functional diversity of alpha-lactalbumins may be essential to maintain health in the intestinal tract and possibly in other organs, and the oleic acid complexes should be worth exploring as a prophylactic and therapeutic tools in oncology.

### REFERENCES

1 Sung, H., Ferlay, J., Siegel, R. L., Laversanne, M., Soerjomataram, I., Jemal, A. & Bray, F. Global cancer statistics 2020: GLOBOCAN estimates of incidence and mortality worldwide for 36 cancers in 185 countries. CA: a cancer journal for clinicians 71, 209-249 (2021).
2 Jasperson, K. W., Patel, S. G. & Ahnen, D. J. APC-associated polyposis conditions. GeneReviews®[Internet] (2017).
3 Vogelstein, B., Fearon, E. R., Hamilton, S. R., Kern, S. E., Preisinger, A. C., Leppert, M., Smits, A. M. & Bos, J. L. Genetic alterations during colorectal-tumor development. New England Journal of Medicine 319, 525-532 (1988).
4 Nieuwenhuis, M. H. & Vasen, H. F. A. Correlations between mutation site in APC and phenotype of familial adenomatous polyposis (FAP): A review of the literature. Critical Reviews in Oncology/Hematology 61, 153-161, doi:doi.org/10.1016/j.critrevonc.2006.07.004 (2007).
5 Moser, A., Luongo, C., Gould, K. A., McNeley, M., Shoemaker, A. & Dove, W. ApcMin: a mouse model for intestinal and mammary tumorigenesis. European Journal of Cancer 31, 1061-1064 (1995).
6 Baan, R., Straif, K., Grosse, Y., Secretan, B., El Ghissassi, F., Bouvard, V., Altieri, A. & Cogliano, V. Carcinogenicity of alcoholic beverages. The Lancet. Oncology 8, 292-293 (2007).
7 Berger, N. A. Vol. 120 935-939 (Wiley Online Library, 2014).
8 Berger, N. A. Obesity and cancer pathogenesis. Annals of the New York Academy of Sciences 1311, 57-76 (2014).
9 Sanchis-Gomar, F., Lucia, A., Yvert, T., Ruiz-Casado, A., Pareja-Galeano, H., Santos-Lozano, A., Fiuza-Luces, C., Garatachea, N., Lippi, G. & Bouchard, C. Physical inactivity and low fitness deserve more attention to alter cancer risk and prognosis fitness and cancer. Cancer Prevention Research 8, 105-110 (2015).
10 Rammer, P., Groth-Pedersen, L., Kirkegaard, T., Daugaard, M., Rytter, A., Szyniarowski, P., Høyer-Hansen, M., Povlsen, L. K., Nylandsted, J., Larsen, J. E. & Jäättelä, M. BAMLET activates a lysosomal cell death program in cancer cells. Molecular Cancer Therapeutics 9, 24-32, doi: 10.1158/1535-7163.Mct-09-0559 (2010).
11 Zhong, S., Liu, S., Chen, S., Liu, H., Zhou, S., Qin, X. & Wang, W. Cytotoxicity and apoptosis induction of bovine alpha-lactalbumin-oleic acid complex in human breast cancer cells. Food Science and Technology Research 21, 103-110 (2015).
12 Mahanta, S. & Paul, S. Stable self-assembly of bovine α-lactalbumin exhibits target-specific antiproliferative activity in multiple cancer cells. ACS Applied Materials & Interfaces 7, 28177-28187 (2015).
13 Sinevici, N., Harte, N., O'Grady, I., Xie, Y., Min, S., Mok, K. H. & O'Sullivan, J. The novel therapeutic potential of bovine α-lactalbumin made lethal to tumour cells (BALMET) and oleic acid in oral squamous cell carcinoma (OSCC). European Journal of Cancer Prevention 30, 178-187 (2021).
14 Moscho, A., Orwar, O., Chiu, D. T., Modi, B. P. & Zare, R. N. Rapid preparation of giant unilamellar vesicles. Proceedings of the National Academy of Sciences 93, 11443-11447 (1996).
15 Stenhouse, G., Fyfe, N., King, G., Chapman, A. & Kerr, K. Thyroid transcription factor 1 in pulmonary adenocarcinoma. Journal of clinical pathology 57, 383-387 (2004).
16 Hu, H., Kang, L., Zhang, J., Wu, Z., Wang, H., Huang, M., Lan, P., Wu, X., Wang, C. & Cao, W. Neoadjuvant PD-1 blockade with toripalimab, with or without celecoxib, in mismatch repair-deficient or microsatellite instability-high, locally advanced, colorectal cancer (PICC): a single-centre, parallel-group, non-comparative, randomised, phase 2 trial. The Lancet Gastroenterology & Hepatology 7, 38-48 (2022).
17 Massari, F., Santoni, M., Ciccarese, C., Santini, D., Alfieri, S., Martignoni, G., Brunelli, M., Piva, F., Berardi, R. & Montironi, R. PD-1 blockade therapy in renal cell carcinoma: current studies and future promises. Cancer treatment reviews 41, 114-121 (2015).
18 Kamphorst, A. O., Pillai, R. N., Yang, S., Nasti, T. H., Akondy, R. S., Wieland, A., Sica, G. L., Yu, K., Koenig, L. & Patel, N. T. Proliferation of PD-1+ CD8 T cells in peripheral blood after PD-1-targeted therapy in lung cancer patients. Proceedings of the National Academy of Sciences 114, 4993-4998 (2017).
19 Rizvi, N. A., Hellmann, M. D., Snyder, A., Kvistborg, P., Makarov, V., Havel, J. J., Lee, W., Yuan, J., Wong, P. & Ho, T. S. Mutational landscape determines sensitivity to PD-1 blockade in non-small cell lung cancer. Science 348, 124-128 (2015).
20 Ferrara, R., Mezquita, L., Texier, M., Lahmar, J., Audigier-Valette, C., Tessonnier, L., Mazieres, J., Zalcman, G., Brosseau, S. & Le Moulec, S. Hyperprogressive disease in patients with advanced non-small cell lung cancer treated with PD-1/PD-L1 inhibitors or with single-agent chemotherapy. JAMA oncology 4, 1543-1552 (2018).
21 Brahmer, J. R., Tykodi, S. S., Chow, L. Q., Hwu, W.-J., Topalian, S. L., Hwu, P., Drake, C. G., Camacho, L. H., Kauh, J. & Odunsi, K. Safety and activity of anti-PD-L1 antibody in patients with advanced cancer. New England Journal of Medicine 366, 2455-2465 (2012).
22 Topalian, S. L., Hodi, F. S., Brahmer, J. R., Gettinger, S. N., Smith, D. C., McDermott, D. F., Powderly, J. D., Carvajal, R. D., Sosman, J. A. & Atkins, M. B. Safety, activity, and immune correlates of anti-PD-1 antibody in cancer. New England Journal of Medicine 366, 2443-2454 (2012).
23 Chrétien, S., Zerdes, I., Bergh, J., Matikas, A. & Foukakis, T. Beyond PD-1/PD-L1 Inhibition: What the Future Holds for Breast Cancer Immunotherapy. Cancers 11, 628 (2019).
24 Han, Y., Liu, D. & Li, L. PD-1/PD-L1 pathway: current researches in cancer. American journal of cancer research 10, 727 (2020).
25 Patsoukis, N., Wang, Q., Strauss, L. & Boussiotis, V. A. Revisiting the PD-1 pathway. Science Advances 6, eabd2712 (2020).
26 Königshoff, M., Balsara, N., Pfaff, E.-M., Kramer, M., Chrobak, I., Seeger, W. & Eickelberg, O. Functional Wnt signaling is increased in idiopathic pulmonary fibrosis. PloS one 3, e2142 (2008).
27 Wong, R. P., Hwang, W. S. & Field, S. K. Familial adenomatous polyposis and lung cancer. Journal of surgical oncology 60, 213-214 (1995).
28 Puthia, M., Storm, P., Nadeem, A., Hsiung, S. & Svanborg, C. Prevention and treatment of colon cancer by peroral administration of HAMLET (human α-lactalbumin made lethal to tumour cells). Gut 63, 131-142 (2014).
29 Hansen, J. S., Thompson, J. R., Hélix-Nielsen, C. & Malmstadt, N. Lipid directed intrinsic membrane protein segregation. Journal of the American chemical society 135, 17294-17297, doi: 10.1021/ja409708e (2013).
30 Horger, K. S., Estes, D. J., Capone, R. & Mayer, M. Films of agarose enable rapid formation of giant liposomes in solutions of physiologic ionic strength. Journal of the American chemical society 131, 1810-1819 (2009).
31 Peruzzi, J., Gutierrez, M. G., Mansfield, K. & Malmstadt, N. Dynamics of hydrogel-assisted giant unilamellar vesicle formation from unsaturated lipid systems. Langmuir 32, 12702-12709 (2016).

1. A complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, for use in the treatment or prevention of cancer; wherein the complex is for administration at a first site and the cancer is at a second site.

2. A complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, for use as a checkpoint inhibitor, particularly an inhibitor of PD-1, or for use in the prevention or treatment, particularly treatment, of PD-L1 positive cancers or other cancers that are susceptible to PD-1 targeting.

3. A complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, for use in the prevention, reduction, or treatment of metastasis.

4. A complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, for use in tumor surveillance and/or altering the tumor environment.

5. A complex for use, according to claim 2, 3 or 4, wherein the complex is for administration at a first site and the cancer is at a second site.

6. A complex comprising a polypeptide having a sequence of a naturally occurring alpha-lactalbumin, or a functional variant thereof; or a peptide of up to 50 amino acids comprising an alpha-helical domain of said polypeptide; for use in the treatment or prevention of metabolic-related conditions, such as insulin resistance, type II diabetes, metabolic syndrome, non-alcoholic fatty acid liver disease, cirrhosis, or high blood pressure.

7. A method of preventing or treating cancer comprising the step of administering an effective amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, or a pharmaceutical composition comprising the complex, to a subject having cancer, or a predisposition to, or an increased likelihood of developing a cancer, wherein the complex or composition is for administration to a first site, and the cancer is at a second site.

8. A method of preventing or treating a cancer that is PD-L1 positive, or is otherwise susceptible to PD-1 targeting, comprising the step of administering an effective amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, or a pharmaceutical composition comprising the complex, to a subject in need thereof.

9. A method of preventing or treating metastatic cancer, comprising the step of administering an effective amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, or a pharmaceutical composition comprising the complex, to a subject in need thereof.

10. A method of tumor surveillance and/or altering the tumor environment, comprising the step of administering an effective amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, or a pharmaceutical composition comprising the complex, to a subject in need thereof.

11. A method according to claim 8, 9 or 10, wherein the wherein the complex or pharmaceutical composition is administered at a first site and the cancer is at a second site.

12. A method of treating metabolic-related conditions, such as insulin resistance, type II diabetes, metabolic syndrome, non-alcoholic fatty acid liver disease, cirrhosis, or high blood pressure, comprising the step of administering an effective amount of a complex comprising a polypeptide having a sequence of a naturally occurring alpha-lactalbumin, or a functional variant thereof; or a peptide of up to 50 amino acids comprising an alpha-helical domain of said polypeptide; and a fatty acid or lipid or salt thereof, or a pharmaceutical composition comprising the complex, to a subject in need thereof.

13. The complex for use according to any of claim 1 to 6, or a method according to any of claim 7 to 12, wherein the complex or pharmaceutical composition is for administration to, or is administered to one of nasal passage, the GI tract, the brain, the lung, the kidney, the vagina, the bladder, the liver, the skin, the breast, the prostate and/or the ovary.

14. The complex for use according to any of claim 1 to 5 or 13, or a method according to any of claim 7 to 10 or 13, wherein the cancer is found in one or more of nasal passage, the GI tract, the brain, the lung, the kidney, the vagina, the bladder, the liver, the skin, the breast, the prostate and/or the ovary.

15. The complex for use according to any of claim 1 to 5, 13 or 14, or a method according to any of claim 7 to 10, 13 or 14, wherein the site of administration of the complex or pharmaceutical composition and the site of the cancer are different.

16. The complex for use or method according to claim 15, wherein the site of administration of the complex or pharmaceutical composition and the site of the cancer are remote from one another.

17. The complex for use according to claim 2, or the method claim 8, wherein the cancer is primary cancer.

18. The complex for use according to claim 2, or the method claim 8, wherein the cancer is a metastasis.

19. The complex for use according to any of claim 2, 3 or 17, or the method according to any of claim 8, 9 or 18, wherein the site of administration of the complex or pharmaceutical composition and the site of the cancer are the same.

20. The complex for use according claim 6, or the method according to claim 12, wherein the metabolic-related condition is secondary to cancer, or is found in a subject having or that has had cancer.

21. The complex for use according claim 6, or the method according to claim 12, wherein the metabolic-related condition is independent of cancer, or is found in a subject that does not have or has not had cancer.

22. The complex for use or method accordingly to any preceding claim, wherein the polypeptide has a sequence of a naturally occurring alpha-lactalbumin, particularly a human or bovine alpha-lactalbumin.

23. The complex for use or method accordingly to any preceding claim, wherein the polypeptide has a sequence of a bovine alpha-lactalbumin.

24. The complex for use or method accordingly to any preceding claim, wherein the alpha-helical domain is the Alpha 1 (residues 1-39) or Alpha 2 (residues 81-123) domain of human alpha-lactalbumin, being of SEQ ID NO 3 or SEQ ID NO 4; KQFTK XELSQLLKDIDGYGGIALPELI XTMFHTSGYDTQ (SEQ ID NO 3) LDDDITDDIM XAKKILDIKGIDYWLAHKALXTEKLEQWL XEKL (SEQ ID NO 4) where X is an amino acid residue other than cysteine.

25. The complex for use or method accordingly to any preceding claim, wherein the functional variant consists of a sequence lacking disulfide bonds.

26. The complex for use or method accordingly to any preceding claim, wherein the functional variant consists of a sequence in which cysteine residues in the native alpha-lactalbumin are changed to other amino acid residues, preferably alanine residues.

27. The complex for use or method accordingly to any preceding claim, wherein the fatty acid or lipid or salt thereof is a fatty acid or salt thereof, particularly oleic acid or an oleate salt.

28. The complex for use or method accordingly to any preceding claim, wherein the polypeptide has the sequence of bovine alpha-lactalbumin and the fatty acid or salt thereof is oleic acid or an oleate salt.

29. The complex for use or method accordingly to any preceding claim, wherein the complex or pharmaceutical composition is in the form of a beverage, a foodstuff, an additive or component for a beverage or foodstuff, or another a nutritional composition.
